(19) United States
(12) Patent Application Publication
(10) Pub. No.: US 2025/0288611 A1
(43) Pub. Date: Sep. 18, 2025

(54) ICAM-1 TARGETED CAR CONSTRUCTS AND METHODS OF TREATMENT

(71) Applicant: AffyImmune Therapeutics Inc., Natick, MA (US)

(72) Inventors: Matt Britz, Burlington, MA (US); Sonal Gupta, Lexington, MA (US); Alyssa Birt, Northborough, MA (US); Josh Boutin, Brookline, MA (US)

(21) Appl. No.: 19/069,125

(22) Filed: Mar. 3, 2025

Related U.S. Application Data
(60) Provisional application No. 63/561,200, filed on Mar. 4, 2024.
(60) Provisional application No. 63/718,025, filed on Nov. 8, 2024.

(51) Int. Cl. A61K 35/17; A61K 31/496; A61K 31/5025; A61K 31/506; A61K 31/517; A61K 40/11; A61K 40/31; A61K 40/42; A61K 45/06; A61K 47/69; A61K 51/12; A61P 35/00; C07K 14/705; C07K 14/72; C07K 14/725; C07K 16/28; C12N 15/86

(52) U.S. Cl. CPC A61K 35/17; A61K 31/496; A61K 31/5025; A61K 31/506; A61K 31/517; A61K 40/11; A61K 40/31; A61K 40/421; A61K 40/4237; A61K 40/4254; A61K 45/06; A61K 47/6901; A61K 51/1203; A61P 35/00; C07K 14/7051; C07K 14/70517; C07K 14/70521; C07K 14/70578; C07K 14/72; C07K 16/2821; C12N 15/86; A61K 2123/00; A61K 2239/13; A61K 2239/17; A61K 2239/21; C12N 2740/15043

(57) ABSTRACT

The present disclosure relates to cell therapy methods for treating solid carcinoma tumors comprising administration of immune cells expressing a chimeric antigen receptor (CAR) comprising a mutant I domain of the α_{L} subunit of human lymphocyte function-associated antigen-1 (LFA-1), which are cytotoxic against solid carcinoma tumors overexpressing ICAM-1 and alleviate on-target, off-tumor toxicities.

### CROSS-REFERENCE TO RELATED APPLICATIONS

The present disclosure is related and claims priority under 35 U.S.C. § 119(e), to U.S. Provisional Patent Application No. 63/561,200, entitled ICAM-1 TARGETED CAR CONSTRUCTS AND METHODS OF TREATMENT, to Matt Britz et al., filed on Mar. 4, 2024, and to U.S. Provisional Patent Application No. 63/718,025, entitled ICAM-1 TARGETED CAR CONSTRUCTS AND METHODS OF TREATMENT, to Matt Britz et al., filed on Nov. 8, 2024, the contents of each of which are hereby incorporated by reference in their entireties, for all purposes.

### SEQUENCE LISTING

The instant application contains a Sequence Listing which has been submitted electronically in XML file format and is hereby incorporated by reference in its entirety. Said XML copy, created on Feb. 28, 2025, is named 115568-19-25US_SL.xml and is 37,983 bytes in size.

### FIELD

The present disclosure relates to ICAM-1 targeted CAR constructs and ICAM-1 targeted CAR-expressing cells and methods of their use in the treatment of cancer.

### BACKGROUND

Adoptive cell transfer therapy is a type of immunotherapy involving ex vivo expansion of autologous or allogeneic immune cells and subsequent infusion into a patient. The immune cells may be modified ex vivo to specifically target malignant cells. Modifications include engineering of T cells to express chimeric antigen receptors (CARs). The advantage of CAR-T technology compared with chemotherapy or therapeutic antibodies is that reprogrammed engineered T cells can proliferate and persist in the patient and work like a living drug.

Cancer therapy utilizing adoptive transfer of chimeric antigen receptor (CAR) T cells has demonstrated remarkable clinical outcomes in hematologic malignancies. However, CAR T cell application to solid tumors has had limited success, partly due to the lack of tumor-specific antigens and an immune-suppressive tumor microenvironment.

The ideal CAR target antigen would be a native, surface-exposed tumor neoantigen that is highly expressed and is undetectable in healthy tissues. However, many commonly targeted solid tumor antigens are also expressed by non-tumor tissues, albeit at lower levels. CAR molecules with high affinity for such antigens can lead to collateral targeting of healthy tissues resulting in on-target, off-tumor toxicity, a major limiting factor to the progress of CAR T cell therapy to date.

Intercellular adhesion molecule 1 (ICAM-1) is a cell surface glycoprotein receptor involved in cell-cell and cell-matrix adhesive interactions. ICAM-1 is overexpressed in several cancers, and the survival rate of patients with high ICAM-1 expression is worse than that of those with low expression.

It is therefore of great interest to develop CARs and CAR-T expressing cells facilitating more selective killing of carcinoma tumor cells, reduced T cell exhaustion and T cell activation, improved in vivo persistence, and reduced systemic toxicities.

### SUMMARY OF THE INVENTION

Previously, it was observed that ICAM-1 is expressed in normal human tissue and neoplastic human tissues and that certain CAR-T cells have the undesirable effect of killing healthy cells and tissue expressing ICAM-1. See e.g., Park S. et al. Sci Rep. 2017 Oct. 30; 7(1):14366; Dustin M L et al, J Immunol. 1986; 137:245-254. The present disclosure features the use of affinity tuned anti-ICAM-CAR-T cells that avoid this problem as reflected in reduced killing of healthy cells while maintaining efficacy against high ICAM-1 expressing carcinoma cells.

In one aspect, the present disclosure provides a method of cell therapy for treating cancer, comprising the steps of administering to a subject with carcinoma a population of CAR-T cells expressing a chimeric antigen receptor (CAR). The CAR comprises from N-terminus to C-terminus: (i) an I domain of the αL subunit of human lymphocyte function-associated antigen-1, (ii) a transmembrane domain, (iii) one or more co-stimulatory domains, and (iv) an activation domain. The I domain comprises amino acids 128-311 of SEQ ID NO: 1 with one or more mutations selected from the group consisting of F265S, I288N, L289G, F292A, F292G, F292S, L295A, I309T, G311C, and combinations thereof. In particular embodiment, the CAR comprises the I domain mutant, F292A. The cancer is a non-small cell lung carcinoma, small cell lung carcinoma, squamous cell lung carcinoma, large cell lung carcinoma, cervical carcinoma, hepatocellular carcinoma, renal carcinoma, bladder carcinoma, head and neck carcinoma, adenocarcinoma thereof, or a squamous cell carcinoma thereof. In addition, the carcinoma overexpresses ICAM-1, and the CAR-T cells bind to and kill carcinoma cells overexpressing ICAM-1.

In an embodiments, the carcinoma is a metastatic carcinoma. In one embodiment, the subject is a patient with non-small cell lung carcinoma. In another embodiment, the subject is a patient with cervical cancer.

In an embodiment, the method comprises intravenously infusing CAR-T cells into a patient at a dose between 10⁶ to 10¹⁰ cells per patient. In another embodiment, the method comprises intravenously infusing CAR-T cells into a patient at a dose between 10⁷-10⁹ cells per patient. In another embodiment, the method comprises intravenously infusing CAR-T cells into a patient at a dose between 10⁸ to 10⁹ cells per patient.

In another embodiment, the transmembrane domain is positioned between the I domain and the one or more co-stimulatory domains and is from a cell surface receptor selected from the group consisting of an alpha, beta, or zeta chain of a T cell receptor, CD8 alpha, CD28, ICOS, GITR, CD3 epsilon, CD45, CD4, CD5, CD8, CD9, CD16, CD22, CD33, CD37, CD64, CD80, CD86, CD134, CD137, CD154, CD271, TNFRSF19, Killer Cell Immunoglobulin-Like Receptor (KIR), and a combination thereof. In an exemplary embodiment, the transmembrane domain comprises an amino acid sequence set forth in any one SEQ ID NOs: 4-7.

In an embodiment, the one or more co-stimulatory domains are from 4-1BB (CD 137), CD28, OX40, ICOS, GITR, CD27, CD30, CD40, DAP 10, DAP12, BAFFR, HVEM, ICAM-1, lymphocyte function-associated antigen-1 (LFA-1), CD2, CD5, CD7, CD287, LIGHT, NKG2C, NKG2D, SLAMF7, NKp80, NKp30, NKp44, NKp46, CD 160, B7-H3, a ligand that specifically binds with CD83, or a combination thereof. In another embodiment, the one or more co-stimulatory domains from CD28, 4-1BB, ICOS-1, CD27, OX-40, GITR, DAP10, or a combination thereof. In another embodiment, the one or more co-stimulatory domains from CD28 or 4-1BB. In another embodiment, the one or more co-stimulatory domains are from CD28 and 4-1BB. In an exemplary embodiment, the costimulatory domain comprises an amino acid sequence set forth in any one of SEQ ID NOs: 8-10.

In an embodiment, the activation domain is from CD3 zeta, common FcR gamma (FCER1G), Fc gamma RIIa, FcR beta, CD3 gamma, CD3 delta, CD3epsilon, CD5, CD22, CD79a, CD79b, CD278 (ICOS), FcεRI, or CD66d. In another embodiment, the activation domain is from CD3 zeta. In an exemplary embodiment, the activation domain comprises the amino acid sequence of SEQ ID NO: 11.

In an embodiment, the CAR further comprises a hinge domain between the I domain and the transmembrane domain. In one embodiment, the hinge domain is from CD8 alpha, CD28, or IgG4. In an exemplary embodiment, the hinge domain comprises an amino sequence set forth in any one of SEQ ID NOs: 12-14.

In an embodiment, the CAR-T cells further express an epitope tag. In an exemplary embodiment, the epitope tag is a c-myc tag comprising the amino acid sequence set forth in SEQ ID NO: 15.

In an embodiment, the CAR-T cells further express one or more additional polypeptides. In one embodiment, the CAR-T cells express human somatostatin receptor 2 (SSTR2) comprising an amino acid sequence set forth in SEQ ID NO: 16 or SEQ ID NO: 17. In another embodiment, the CAR-T cells express an immune checkpoint inhibitor. In another embodiment, an immune checkpoint inhibitor is administered in combination with the CAR-T cells. In one embodiment, the CAR-T cells are administered in combination with a PD-1 inhibitor. In another embodiment, the CAR-T cells are administered in combination with an anti-PD-1 antibody, such as pembrolizumab, ipilimumab, nivolumab, or atezolizumab.

In an embodiment, the CAR-T cells express a cytokine or are co-administered with an anti-inflammatory cytokine, such as IL-4, IL-10, IL-11, IL-13, or a combination thereof.

In another embodiment, the CAR-T express or are co-administered with a proinflammatory cytokine antagonist. In one embodiment, the proinflammatory cytokine antagonist targets a cytokine selected from the group consisting of IL2, IL1α, IL1β, IL-5, IL-6, IL-7, IL-8, IL-9, IL-12, IL-15, IL-17, IL-18, IL-21, IL-23, sIL-1RI, sIL-2Rα, sIL6R, IFNα, IFNβ, IFNγ, MIPα, MIPβ, CSF1, LIF, G-CSF, GM-CSF, CXCL10, CCL5, eotaxin, TNF, MCP1, MIG, RAGE, CRP, angiopoietin-2, VWF, TGFα, VEGF, EGF, HGF, FGF, perforin, granzyme, and ferritin. In another embodiment, the proinflammatory cytokine antagonist targets IFNγ, IL-1, IL-6, IL-12, GM-CSF, or a combination thereof.

In an embodiment, one or more therapeutic antibodies are administered in combination with the CAR-T cells. In an embodiment, the therapeutic antibody is selected from the group consisting of abagovomab, adecatumumab, afutuzumab, alemtuzumab, altumomab, amatuximab, anatumomab, arcitumomab, bavituximab, bectumomab, bevacizumab, bivatuzumab, blinatumomab, brentuximab, cantuzumab, catumaxomab, cetuximab, citatuzumab, cixutumumab, clivatuzumab, conatumumab, daratumumab, drozitumab, duligotumab, dusigitumab, detumomab, dacetuzumab, dalotuzumab, ecromeximab, elotuzumab, ensituximab, ertumaxomab, etaracizumab, farietuzumab, ficlatuzumab, figitumumab, flanvotumab, futuximab, ganitumab, gemtuzumab, girentuximab, glembatumumab, ibritumomab, igovomab, imgatuzumab, indatuximab, inotuzumab, intetumumab, ipilimumab, iratumumab, labetuzumab, lexatumumab, lintuzumab, lorvotuzumab, lucatumumab, mapatumumab, matuzumab, milatuzumab, minretumomab, mitumomab, moxetumomab, namatumab, naptumomab, necitumumab, nimotuzumab, nofetumomab, ocaratuzumab, ofatumumab, obinutuzumab, olaratumab, onartuzumab, oportuzumab, oregovomab, panitumumab, parsatuzumab, patritumab, pemtumomab, pertuzumab, pintumomab, pritumumab, racotumomab, radretumab, rilotumumab, rituximab, robatumumab, satumomab, sibrotuzumab, siltuximab, simtuzumab, solitomab, tacatuzumab, taplitumomab, tenatumomab, teprotumumab, tigatuzumab, tositumomab, trastuzumab, tucotuzumab, ublituximab, veltuzumab, vorsetuzumab, votumumab, zalutumumab, CC49, and combinations thereof.

In an embodiment, the subject is additionally administered or infused with a second population of CAR-T cells expressing a second CAR. In one embodiment, the second CAR comprises a c-Met binding domain, a CEACAM-5 binding domain, or an EpCAM binding domain.

In another aspect, the CAR-T cells express SSTR2 and the method further comprises the steps of: (i) incubating the CAR-T cells with a radioactive label that binds to SSTR2 before administration into a patient with the carcinoma, (ii) intravenously infusing the CAR-T cells labeled in step (i) into the patient, and (iii) subjecting the patient to PET/CT imaging to monitor labeled CAR-T cells and their in vivo distribution in the patient. In an exemplary embodiment, the radioactive label is ⁶⁸Ga-DOTATOC or ⁶⁸Ga-DOTATATE.

In an embodiment, the method further includes the step of expanding the CAR-T cells in the presence of a tyrosine kinase inhibitor capable of inhibiting TCR signaling and/or CAR signaling before administration of the CAR-T cells in the subject. In one embodiment, the tyrosine kinase is dasatinib, ponatinib, saracatinib, bosutinib, nilotinib, or a combination thereof. In another embodiment, the tyrosine kinase is dasatinib.

In an embodiment, the cell therapy method includes the step of transfecting an expression vector encoding a CAR described herein to produce the CAR-T cells prior to the administration step. In one embodiment, the expression vector is a virus vector. In another embodiment, the virus vector is a lentivirus. In one embodiment, the expression vector comprises from 5′ and 3, coding sequences encoding: (i) an I domain according to the present disclosure; (ii) a CD8 hinge region; (iii) a CD28 transmembrane domain; (iv) a CD28 costimulatory domain; (v) a 4-1BB costimulatory domain; (vi) a CD3ζ activation domain; and optionally an epitope tag, such as a c-myc tag. In one embodiment, the epitope tag is upstream of the I domain.

In an embodiment, the expression vector further comprises one or more self-cleaving 2A peptide coding sequences or IRES elements. In one embodiment, the self-cleaving 2A peptide coding sequences are from porcine teschovirus-1 (P2A), equine rhinitis A virus (E2A), thosea asigna virus (T2A), or foot-and-mouth disease virus (F2A), or a combination thereof. In some embodiments, the self-cleaving 2A peptide coding sequences or IRES elements are downstream of the activation domain.

In an embodiment, the expression vector further encodes one or more additional polypeptides. In some embodiments, the additional polypeptides are downstream of the self-cleaving 2A peptide coding sequences or IRES elements. In some embodiments, the one or more additional polypeptides comprise SSTR2. In an embodiment, the expression vector comprises a nucleic acid comprising the nucleotide sequence set forth in SEQ ID NO: 25 or SEQ ID NO: 26.

In another embodiment, the expression vector encodes an immune checkpoint inhibitor, a cytokine, or both. In an embodiment, the checkpoint inhibitor is a PD-1 inhibitor. In another embodiment the cytokine is IL-12.

In some embodiments, the cell therapy method further includes the step of administering a therapy to reduce tumor burden in the subject prior to the cell therapy against cancer. In some embodiments, the therapy is a chemotherapy, an immunotherapy, a radiotherapy, or a surgery. In some embodiments, prior to administration of the CAR-T cells, the subject receives a lymphodepleting treatment to condition the subject for administration of the CAR-T cells. In some embodiments, the lymphodepleting treatment comprises administering to the subject one or more of fludarabine and cyclophosphamide. In other embodiments, the lymphodepleting treatment comprises administering to the subject bendamustine alone, or in combination with other suitable treatments.

In an embodiment, the cell therapy method further comprises administering to the subject a tyrosine kinase inhibitor capable of inhibiting TCR signaling and/or CAR signaling. In some embodiments, the tyrosine kinase is selected from the group consisting of dasatinib, ponatinib, saracatinib, bosutinib, nilotinib, and combinations thereof. In one embodiment, the tyrosine kinase inhibitor is dasatinib.

In some embodiments, the tyrosine kinase inhibitor is administered for a period of time sufficient to restore at least partial T cell function and then discontinued. In some embodiments, the tyrosine kinase inhibitor is administered continuously. In some embodiments, the tyrosine kinase inhibitor is administered intermittently. In some embodiments, the tyrosine kinase inhibitor administered iteratively for purposes of facilitating periods of T cell inactivation during pharmaceutical composition administration and periods of T cell activation during absence of pharmaceutical composition administration. In some embodiments, the tyrosine kinase inhibitor is administered intermittently so that the tyrosine kinase concentration is maintained below a threshold level required to block CAR-T cell function.

In some embodiments, the CAR-T cells express SSTR2, and the subject is administered a safety switch comprising an SSTR2-targeted cytotoxic conjugate if unacceptable toxicity from the CAR-T cells is identified, the conjugate comprising a cytotoxic agent coupled to an SSTR2 targeting moiety by a linker. In some embodiments, the targeting moiety is somatostatin, a somatostatin analog, octreotide, lanreotide, lutathera (¹⁷⁷Lu-DOTATATE), ⁹⁰Y-DOTATOC, Tyr3-octreotate (TATE), vapreotide, cyclo(AA-Tyr-DTrp-Lys-Thr-Phe) where AA is α-N-Me lysine or N-Me glutamic acid, pasireotide, lanreotide, seglitide, or an anti-SSTR2 antibody.

In some embodiments, the cytotoxic agent is a maytansinoid, a camptothecin, a dolastatin, an aurastatin, a pyrrolobenzodiazepine (PBD), a calicheamicin, a duocarmycin, a tubulolysin, an analogue thereof, or a combination thereof. In some embodiments, the cytotoxic agent is deruxtecan, exatecan, SN-38, monomethyl auristatin E (MMAE), monomethyl auristatin F (MMAE), Auristatin F-hydroxypropylamide, Aur0101, emtansine (DM1), Maytansinoid DM4, duocarmycin, duostatin-5, or a combinations thereof.

In some embodiments, the linker is a cleavable linker selected from the group consisting of maleimidocaproyl-L-valine-L-citrulline-p-aminobenzoyl carbamate (mc-VC-PABC) and maleimido-dPEG8-L-valine-L-alaline-p-aminobenzoyl carbamate (M-dPEG8-VA-PABC). In some embodiments, the linker is a non-cleavable linker selected from the group consisting of maleimidocaproyl (MC), succinimidyl-4-(N-maleimidomethyl)cyclohexane-1-carboxylate (SMCC), and thioether-containing linker.

In some embodiments, the cytotoxic conjugate has a drug-to-antibody (DAR) ratio between 2-10. In some embodiments, the cytotoxic conjugate is PEN-221.

The details of one or more embodiments of the invention are set forth in the description below. Other features or advantages of the present invention will be apparent from the following drawings and detailed description of several embodiments, and also from the appended claims.

### BRIEF DESCRIPTION OF THE FIGURES

FIG. 1 is a schematic depiction of a CAR expression construct used in the Examples described herein.

FIGS. 2A-2B are graphs showing relative levels of ICAM-1 expression by immunohistochemistry of non-small cell lung adenocarcinoma (FIG. 2A) and squamous cell carcinoma (FIG. 2B) tumor arrays across various differentiation states. FIG. 2C is a table summarizes the results in FIGS. 2A-2B.

FIG. 3A is a graph showing relative levels of ICAM-1 expression by immunohistochemistry of a squamous cell cervical tumor array across various differentiation states. FIG. 3B summarizes the results in FIG. 3A.

FIG. 4A is a graph showing % cytotoxicity as a function of time using a human non-small cell lung squamous carcinoma cell line (H226) co-cultured with ICAM-1 CAR-T cells or non-transduced T cells (NT). FIG. 4B is a histogram showing expression of ICAM-1 in H226 cells by flow cytometry following surface staining with an anti-ICAM-1 phycoerythrin (PE-A) conjugate (right peak) or staining with anti-IgG1 isotype control conjugated to phycoerythrin (PE-A-).

FIG. 5A is a graph showing % cytotoxicity as a function of time using a human non-small cell lung adenocarcinoma cell line (H441) co-cultured with ICAM-1 CAR-T cells or non-transduced T cells (NT). FIG. 5B is a histogram showing expression of ICAM-1 in H441 cells by flow cytometry following surface staining with an anti-ICAM-1 phycoerythrin (PE-A) conjugate (right peak) or staining with anti-IgG1 isotype control conjugated to phycoerythrin (PE-A-).

FIG. 6A is a graph showing % cytotoxicity as a function of time using a human squamous cell cervical carcinoma cell line (SW756) co-cultured with ICAM-1 CAR-T cells or non-transduced T cells (NT). FIG. 6B is a histogram showing expression of ICAM-1 in SW756 cells by flow cytometry following surface staining with an anti-ICAM-1 phycoerythrin (PE-A) conjugate (right peak) or staining with anti-IgG1 isotype control conjugated to phycoerythrin (PE-A-).

FIG. 7A is a graph showing % cytotoxicity as a function of time using a human cervical carcinoma cell line (HeLa) co-cultured with ICAM-1 CAR-T cells or non-transduced T cells (NT). FIG. 7B is a histogram showing expression of ICAM-1 in HeLa cells by flow cytometry following surface staining with an anti-ICAM-1 phycoerythrin (PE-A) conjugate (right peak) or staining with anti-IgG1 isotype control conjugated to phycoerythrin (PE-A-).

FIG. 8A is a graph showing % cytotoxicity as a function of time using a human hepatocellular carcinoma cell line (HepG2) co-cultured with ICAM-1 CAR-T cells or non-transduced T cells (NT). FIG. 8B is a histogram showing expression of ICAM-1 in HepG2 cells by flow cytometry following surface staining with an anti-ICAM-1 phycoerythrin (PE-A) conjugate (right peak) or staining with anti-IgG1 isotype control conjugated to phycoerythrin (PE-A-).

FIG. 9 is a graph showing % cytotoxicity as a function of time using a human hepatocellular carcinoma cell line (SNU423) co-cultured with ICAM-1 CAR-T cells or non-transduced T cells (NT).

FIG. 10 is a graph showing % cytotoxicity as a function of time using a human squamous cell pharyngeal carcinoma cell line (FaDu) co-cultured with ICAM-1 CAR-T cells or non-transduced T cells (NT).

FIG. 11A shows images of human non-small cell lung squamous carcinoma (H226) xenografts in mice after treatment with ICAM-1 CAR-T cells (Affy80) or in mice administered vehicle only (PBS). FIG. 11B is a graph showing the decrease in total flux (imaging signal to total flux in photons/sec (p/s)) following administration of ICAM-1 CAR-T (Affy80) cells into the H226 xenograft mice.

FIG. 12A shows images of human non-small cell adenocarcinoma (H441) xenografts in mice after treatment with ICAM-1 CAR-T cells (Affy80) or in mice administered vehicle only (PBS) FIG. 12B is a graph showing the decrease in total flux (imaging signal to total flux in photons/sec (p/s)) following administration of ICAM-1 CAR-T (Affy80) cells into the H441 xenograft mice.

FIG. 13A shows images of human cervical carcinoma (SW756) xenografts in mice after treatment with ICAM-1 CAR-T cells (Affy80) or in mice administered vehicle only (PBS). FIG. 13B is a graph showing the decrease in total flux (in p/s) following administration of ICAM-1 CAR-T (Affy80) cells into the SW756 xenograft mice.

FIG. 14A shows images of human HeLa cell xenografts in mice after treatment with ICAM-1 CAR-T cells (Affy80) or in mice administered vehicle only (PBS) FIG. 14B is a graph showing the decrease in total flux (in p/s) following administration of ICAM-1 CAR-T (Affy80) cells into the HeLa xenograft mice.

FIG. 15 is a graph showing % cytotoxicity as a function of time using primary human lung epithelial cells co-cultured with high affinity c-Met CAR-T cells, (positive control), low affinity ICAM-1 CAR-T cells (Affy80), or nonspecific CAR-T cells (negative control).

FIGS. 16-21 describe the in vivo study described in Example 10 and results thereof.

### DETAILED DESCRIPTION OF THE INVENTION

The present disclosure is based on the unexpected discovery that chimeric antigen receptor (CAR)-T cells expressing an affinity-tuned I domain of the αL subunit of human lymphocyte function-associated antigen-1 (LFA-1) with reduced affinity for ICAM-1 can effectively kill certain ICAM-1 overexpressing carcinoma cells, while avoiding the undesirable effect of killing healthy cells and tissue.

### I. Chimeric Antigen Receptor with Reduced Affinity for ICAM-1

One aspect of the present disclosure provides a method of cell therapy for treating solid carcinoma tumors. The method comprises administering to subject with a carcinoma a population of CAR-expressing immune cells (e.g., CAR-T cells) expressing a chimeric antigen receptor (CAR). The CAR comprises from N-terminus to C-terminus: (i) an I domain of the αL subunit of human lymphocyte function-associated antigen-1, (ii) a transmembrane domain, (iii) one or more co-stimulatory domains, and (iv) an activation domain. The solid carcinoma tumors for treatment according to the methods disclosed herein include non-small cell lung carcinomas, small cell lung carcinomas, squamous cell lung carcinomas, large cell lung carcinomas, cervical carcinomas, hepatocellular carcinomas, renal carcinomas, bladder carcinomas, head and neck carcinomas, as well as adenocarcinomas and squamous cell carcinomas thereof. The carcinoma overexpresses ICAM-1 and upon binding of the CAR-expressing immune cells to ICAM-1 expressed on the surface of the carcinoma, immune responses are triggered and tumor cells are killed.

### (A) Affinity-Tuned ICAM-1 Binding Domain

In an embodiment, a CAR construct comprises an affinity-tuned ICAM-1 binding domain with reduced affinity for the ICAM-1. Such a construct may be referred to herein as an “ICAM-1 CAR” or an “Affy80-CAR”. In an embodiment, the affinity-tuned ICAM-1 binding domain comprises an I domain of the αL subunit of human lymphocyte function-associated antigen-1. In an embodiment, the I domain comprises amino acids 128-311 of SEQ ID NO: 1 with one or more mutations selected from the group consisting of F265S, I288N, L289G, F292A, F292G, F292S, L295A, I309T, G311C, and combinations thereof. The corresponding wild-type I domain comprises the amino acid sequence set forth in SEQ ID NO: 2. In one embodiment, the CAR comprises the I domain mutant, F292A, which comprises the amino acid sequence set forth in SEQ ID NO: 3.

In one embodiment, the I domain comprises an amino acid sequence at least 95% identical to the amino acids 128-311 of SEQ ID NO: 1. In a preferred embodiment, the I domain comprises an amino acid sequence at least 98% identical to the amino acids 128-311 of SEQ ID NO: 1. In a more preferred embodiment, the I domain comprises an amino acid sequence at least 99% identical to amino acids 128-311 of SEQ ID NO: 1.

The affinity-tuned ICAM-1 CARs possess binding affinities in the micromolar range (e.g., about 1-200 μM K_{D}), which minimize off-tumor toxicity against basally expressed antigens in normal tissues and increase therapeutic index. See U.S. Pat. No. 10,428,136. In an embodiment, the reduced affinity I domain includes a mutation in F292A. Compared to the binding affinity (i.e., K_{D}) of 1.5 mM for wild-type I domain binding to ICAM-1, the binding affinity for F292A has been reported to exhibit a ICAM-1 of 20 μM. Id. Other I domain mutants for use in the present invention include I288N (K_{D}=202 μM), I309T (K_{D}=127 μM), L295A (K_{D}=37 μM), F292S (K_{D}=1.24 μM), L289G (K_{D}=196 nM), F292G (K_{D}=119 μM), F265S (K_{D}=145 nM), F265S/F292G (K_{D}=6 nM), F265S/F292G/G311C (K_{D}=1 nM), and combinations thereof as described in U.S. Pat. No. 10,428,136, the disclosures of which are expressly incorporated by reference herein.

In some embodiments, a mutant I domain is utilized, which has an affinity for ICAM-1 (K_{D}) between about 1 μM and about 200 μM, between about 1 μm and about 150 μM, between about 1 μM and about 120 μM, between about 1 μM and about 40 μM, between about 20 μM and 150 μM, between 20 μM and about 120 μM, between about 20 μM and about 40 μM, between about 1 nM and about 1 μM, between about 1 nM and about 200 nM, between about 1 nM and about 120 nM, between about 1 nM and 10 nM, between about 100 nM and 200 nM, or any range thereof, or any range between the individual mutant K_{D} values set forth above.

### (B) Other CAR Components

In addition to the affinity tuned ICAM-1 binding domain (I domain) discussed above, an ICAM-1 CAR construct disclosed herein further comprises a transmembrane domain, one or more costimulatory domains, and an activation domain comprising one or more ITAMs (as described below), such as the CD3ζ signaling domain (which contains 3), or a combination thereof.

The transmembrane domain facilitates insertion of the CAR into the cell membranes and can be inserted between the binding domain and a costimulatory domain. In some embodiments, the transmembrane domain can be inserted between a hinge region and the co-stimulatory domain. It will be appreciated that any transmembrane domain commonly used in CAR constructs can be used here. In some embodiments, the transmembrane domain is obtained from a suitable cell-surface receptor, such as the transmembrane domain of a cell surface receptor of the alpha, beta, or zeta chain of the T cell receptor, CD28, CD3 epsilon, CD45, CD4, CD5, CD8, CD9, CD16, CD22, CD33, CD37, CD64, CD80, CD86, CD134, CD137, CD154, CD271, TNFRSF19, and killer cell immunoglobulin-like receptor (KIR). In certain embodiments, the transmembrane membrane domain is from CD8 alpha, CD28, ICOS, or GITR, and optionally comprises an amino acid sequence set forth in SEQ ID NOs: 4-7, respectively.

The ICAM-1 CARs disclosed herein comprise one or more costimulatory domains. Costimulatory domains typically enhance and/or alter the nature of the response to activation of the activation domain. Co-stimulatory domains suitable for use in the CARs of the present disclosure are typically receptor-derived polypeptides. In some embodiments, the co-stimulatory domains homodimerize. In some embodiments, the co-stimulatory domain may be the intracellular portion of a transmembrane protein (i.e., the co-stimulatory domain may be derived from the transmembrane protein). In exemplary embodiments, the costimulatory domains are from 4-1BB (CD 137), CD28, OX40, ICOS, GITR, DAP 10, DAP12, CD27, CD30, CD40, BAFFR, HVEM, ICAM-1, LFA-1 (CD11a/CD18), CD2, CD5, CD7, CD287, LIGHT, NKG2C, NKG2D, SLAMF7, NKp80, NKp30, NKp44, NKp46, CD 160, B7-H3, and a ligand that specifically binds with CD83. In an embodiment, the one or more co-stimulatory domains from CD28, 4-1BB, ICOS-1, CD27, OX-40, GITR, DAP10, or a combination thereof. In another embodiment, the one or more co-stimulatory domains from CD28 or 4-1BB. In another embodiment, the one or more co-stimulatory domains are from CD28 and 4-1BB. In an exemplary embodiment, one or more costimulatory domains comprises an amino acid sequence set forth in any one of SEQ ID NOs: 8-10.

The ICAM-1 CARs disclosed herein further comprises an activation domain. As used herein, the term “activation domain” refers to an intracellular signaling domain that can trigger the production of one or more cytokines upon activation; increases antibody-dependent cellular cytotoxicity (ADCC) and cell death; and/or increased activation and/or proliferation of CD8⁺ T cells, CD4⁺ T cells, natural killer T cells, γδ T cells, and/or neutrophil proliferation. In some embodiments, the activation domain comprises at least one (e.g., 1, 2, 3, 4, 5, 6, etc.) immunoreceptor tyrosine-based activation motif (ITAM or ITAMa) with the sequence Yxx[L/I]x₍₆₋₈₎Yxx[L/I]) present in the cytoplasmic tail of an immune signaling receptor or associated subunit to induce cell signaling. The ITAM domains for use in the CARs disclosed herein can include signaling domains from several types of immune signaling receptors, including CD3, B7 family costimulatory intracellular signaling proteins such as molecules and tumor necrosis factor receptor (TNFR) superfamily receptors; signaling domains used by NK and NKT cells, such as NKp30 (B7-H6), DAP12, NKG2D, NKp44, NKp46, DAP10, and CD3 zeta; and signaling domains from ITAM-containing human immunoglobulin receptors, such as FcαRI, FcγRI, FcγRIIA, FcγRIIIA, FcγRIIC, and FcRL5. Thus, in certain embodiments, the activation domain is from CD3 zeta, Fc epsilon receptor gamma (FCER1G), Fc gamma RIIa, FcR beta, CD3 gamma, CD3 delta, CD3epsilon, CD5, CD22, CD79a, CD79b, CD278 (also known as “ICOS”), FcεRI, or CD66d. In an exemplary embodiment, the activation domain is from CD3 zeta and comprises the amino acid sequence of SEQ ID NO: 11.

In some embodiments, the ICAM-1 CAR further comprises a hinge between the I domain and the transmembrane domain. In this orientation, the hinge domain provides additional distance between the antigen binding domain and the cell membrane surface on which the CAR is expressed. In some examples, the hinge domain may be from CD28, CD8, or an IgG, such as IgG1 or IgG4. In some embodiments, the hinge domain comprises an amino sequence set forth in any one of SEQ ID NOs: 12-14.

In one embodiment, the ICAM-1 CAR comprises a mutant I domain of LFA-1, a CD8 hinge region, a CD28 transmembrane domain, a CD28 co-stimulatory domain, a 4-1BB co-stimulatory domain, and a CD3ζ signaling domain. In an exemplary embodiment, such an ICAM-1 CAR comprises the amino acid sequence set forth in SEQ ID NO: 27. In another embodiment, the CAR is expressed as an ICAM-1 CAR fusion protein comprising an a C-terminal SSTR2 polypeptide fused in-frame to the CAR via a P2A element and comprising the amino acid sequence set forth in SEQ ID NO: 28. In another embodiment, the CAR is expressed as an ICAM-1 CAR fusion protein comprising an N-terminal signal peptide, a c-myc tag and a C-terminal SSTR2 polypeptide fused in-frame to the CAR via a P2A element and comprising the amino acid sequence set forth in SEQ ID NO: 29, a mature, membrane-bound form (without signal peptide) comprising the amino acid sequence set forth in SEQ ID NO: 30 or a mature, membrane-bound form without the c-myc tag comprising the amino acid sequence set forth in SEQ ID NO: 31.

In some embodiments, the ICAM-1 CAR may include a second extracellular biding domain in the form of a bispecific ICAM-1-CAR to provide improved specificity. In some embodiments, the present disclosure further contemplates a multi-specific ICAM-1-CAR targeting three of more antigens. In some embodiments, the second binding domain comprises a CEACAM-5 binding domain, a c-MET binding domain, or an EpCAM binding domain.

In some instances, the additional binding domain is an antibody or single-chain antibody fragment (scFv) comprising V_{H} and V_{L} regions connected by a peptide linker. In some embodiments, the scFv has a V_{H}→V_{L} orientation. In other embodiments, the scFv has a V_{L}→V_{H} orientation. In some embodiments, the anti-ICAM-1 scFv comprises a linker between the V_{H} and V_{L} regions. Peptide linkers used in scFv constructs are well known in the art and include, for example, the amino acid sequence (GxS)n in which x is an integer between 1-6, inclusive, and n is an integer between 1-10, inclusive. In some embodiments, the linker comprises the amino acid sequence (GGGS)n in which n is an integer between 1-10, inclusive (SEQ ID NO: 19). In one embodiment, the linker comprises the amino acid sequence multimer GGGGSGGGGSGGGGS (SEQ ID NO: 20).

The V_{H} and V_{L} regions are further subdivided into regions of hypervariability, also known as “complementarity determining regions” (“CDR”), interspersed with regions that are more conserved, which are known as “framework regions” (“FR”). Each V_{H} and V_{L} is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the Chothia definition, the AbM definition, and/or the contact definition, all of which are well known in the art. See, e.g., Kabat, E. A., et al. (1991) Sequences of Proteins of Immunological Interest, Fifth Edition, U.S. Department of Health and Human Services, NIH Publication No. 91-3242, Chothia et al., (1989) Nature 342:877; Chothia, C. et al. (1987) J. Mol. Biol. 196:901-917, Al-lazikani et al (1997) J. Molec. Biol. 273:927-948; and Almagro, J. Mol. Recognit. 17:132-143 (2004). See also the Human Genome Mapping Project Resources at the Medical Research Council in the United Kingdom and the antibody rules described at the Bioinformatics and Computational Biology group website at University College London.

In some embodiments, the V_{H} and V_{L} regions are “humanized.” Humanized variable regions (or antibodies) are derived from chimeric immunoglobulins, human immunoglobulins, immunoglobulin chains, or antigen-binding fragments thereof in which minimal sequences are derived from a non-human immunoglobulin. In some embodiments, the humanized variable regions (or antibodies) comprise residues from CDRs of a non-human species (donor antibody) such as mouse, rat, or rabbit having the desired specificity, affinity, and capacity, where the framework region (FR) residues from the non-human species are replaced by corresponding human residues. Furthermore, the humanized binding region may comprise residues that are found neither in the recipient antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of human immunoglobulin consensus sequences.

In some embodiments, an ICAM-1 binding domain or secondary binding domain may comprise a single domain antibody (sdAb), such as a VHH fragment, a single chain Fab fragment, a single chain Fab′ fragment, or a ICAM-1 binding peptide.

In some embodiments, the secondary binding domain targets a tumor-associated antigen (TAA), particularly one that is overexpressed in solid tumors and metastatic tumors of non-small cell lung carcinoma, small cell lung carcinoma, squamous cell lung carcinoma, large cell lung carcinoma, cervical carcinoma, hepatocellular carcinoma, renal carcinoma, bladder carcinoma, head and neck carcinoma, adenocarcinomas thereof, and squamous cell carcinomas thereof. Exemplary solid tumor antigen targets for the second binding domain may include but are not limited to target antigens selected from EpCAM, CEACAM5, c-Met, EGFRvIII, mesothelin, CS-1, GD2, Tn Ag, PSMA, TAG72, CD44v6, CEA, KIT, IL-13Ra2, GD3, CD171, IL-1Ra, PSCA, VEGFR2, Lewis Y, CD24, PDGFR-beta, SSEA-4, folate receptor alpha, ERBB2, Her2/neu, MUC1, EGFR, NCAM, Ephrin B2, CAIX, LMP2, sLe, HMWMAA, o-acetyl-GD2, folate receptor beta, TEM1/CD248, TEM7R, FAP, Legumam, HPV E6 or E7, CLDN6, TSHR, GPRC5D, ALK, Polysialic acid, PLACl, globoH, NY-BR-1, UPK2, HAVCR1, ADRB3, PANX3, GPR20, Ly6k, OR51E2, TARP, and GFRa4.

In some embodiments, the CAR components may be variants of their naturally occurring counterparts. In some embodiments, any of the other CAR components may contain an amino acid sequence at least 80% (e.g., at least 85%, 90%, 95%, 98%, 99% or above) identical to its natural counterpart. In a preferred embodiment, the CAR components are at least 95% identical to the natural counterpart. In more preferred embodiments, the CAR components are at least 98% identical to the natural counterpart. In even more preferred embodiments, the CAR components are at least 99% identical to the natural counterpart.

As used herein, the “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

In some embodiments, the costimulatory or activation domain contain up to 15 (e.g., up to 12, 10, 8, 6, 5, 4, 3, 2, or 1) amino acid residue substitutions relative to the wild-type counterpart. In some examples, the amino acid residue substitutions are conservative amino acid residue substitutions. As used herein, a “conservative amino acid substitution” refers to an amino acid substitution that does not alter the relative charge or size characteristics of the protein in which the amino acid substitution is made. Variants can be prepared according to routine methods for altering polypeptide sequences known to those skilled in the art Conservative substitutions of amino acids may include substitutions within the following groups: ((a) A→G, S; (b) R→K, H; (c) N→Q, H; (d) D→E, N; I C→S, A; (f) Q→N; (g) E→D, Q; (h) G→A; (i) H→N, Q; (j) I→L, V; (k) L→I, V; (l) K→R, H; (m) M→L, I, Y; (n) F→Y, M, L; (o) P→A; (p) S→T; (q) T→S; I W→Y, F; (s) Y→W, F; and (t) V→I, L.

### II. Adoptive CAR-T Cell Therapy

### (A) Method of Cell Therapy

In one aspect, an immune cell population comprising the ICAM-1 CAR modified immune cells described herein may be used in an adoptive immune cell therapy (e.g., CAR-T) for treating the solid tumor carcinomas overexpressing of ICAM-1 as described herein. In an embodiment, a cell therapy method for treating cancer comprises administering to a subject in need thereof, a population of immune cells comprising genetically engineered CAR-expressing cells (e.g., T cells) described herein in an amount suitable for treating the carcinoma.

The cancer for treatment is a solid carcinoma tumor selected from the group consisting of a non-small cell lung carcinoma, small cell lung carcinoma, squamous cell lung carcinoma, large cell lung carcinoma, cervical carcinoma, hepatocellular carcinoma, renal carcinoma, bladder carcinoma, head and neck carcinoma, an adenocarcinoma thereof, or a squamous cell carcinoma thereof. In addition, the carcinoma overexpresses ICAM-1, and the CAR T cells bind to and kill carcinoma cells overexpressing ICAM-1. In one particular embodiment, the subject is a patient with non-small cell lung carcinoma. In another embodiment, the subject is a patient with cervical carcinoma.

To practice the therapeutic methods described herein, an effective amount of an immune cell population comprising the genetically modified CAR-expressing immune cells described herein may be administered to a subject in need of cancer treatment via a suitable route of administration (e.g., intravenous infusion). One or more of the immune cell populations may be mixed with a pharmaceutically acceptable carrier to form a pharmaceutical composition prior to administration, which is also within the scope of the present disclosure.

The immune cells may be autologous to the subject, e.g., obtained from the subject in need of the treatment, modified to express the ICAM-1 CAR construct and optionally one or more additional exogenous gene products. The resultant modified immune cells can then be administered to the same subject. Administration of autologous cells to a subject may result in reduced rejection of the immune cells as compared to administration of non-autologous cells. Alternatively, the immune cells can be allogeneic cells, e.g., the cells are obtained from a first subject, modified as described herein and administered to a second subject that is different from the first subject but of the same species. For example, allogeneic immune cells may be derived from a human donor and administered to a human recipient who is different from the donor.

The subject to be treated may be a mammal (e.g., human, mouse, pig, cow, rat, dog, guinea pig, rabbit, hamster, cat, goat, sheep, or monkey) suffering from cancer, particularly a human patient with a solid carcinoma tumor characterized by overexpression of ICAM-1 as described herein. The term “an effective amount” as used herein refers to the amount of each active agent required to confer therapeutic effect on the subject, either alone or in combination with one or more active agents. Effective amounts may vary, as recognized by those skilled in the art, depending on the particular condition being treated, the severity of the condition, toxicity consideration, previous trial results, individual patient parameters including age, physical condition, size, gender and weight, the duration of treatment, route of administration, excipient usage, co-usage (if any) with other active agents and like factors within the knowledge and expertise of the health practitioner. The quantity to be administered depends on the subject to be treated, including, for example, the capacity of the individual's immune system to produce a cell-mediated immune response. Effective amounts of the genetically engineered CAR-T cells required to be administered depend on the judgment of the practitioner. However, suitable dosage ranges are readily determinable by one skilled in the art.

The term “treating” as used herein refers to the application or administration of a cell composition to a subject with cancer with the purpose to cure, heal, alleviate, relieve, alter, remedy, ameliorate, improve, affect progression of the cancer and its symptoms.

An effective amount of the immune cells may be administered to a human patient in need of the treatment via a suitable route, e.g., intravenous infusion. In some instances, about 1×10⁷ to about 1×10¹¹ CAR-T cells may be given to a human patient (e.g., a leukemia patient, a lymphoma patient, or a multiple myeloma patient). In some examples, a human patient may receive multiple doses of the immune cells. For example, the patient may receive two doses of the immune cells on two consecutive days. Additionally, dosing may also occur up to 1 year after initial administration. In some instances, the first dose is the same as the second dose. In other instances, the first dose is lower than the second dose, or vice versa.

In an embodiment, the method comprises intravenously infusing CAR-T cells into a subject or patient in an amount between 10⁶ to 10¹¹ T cells/patent. In another embodiment, the method comprises intravenously infusing CAR-T cells into a patient at a dose between 10⁷ to 10¹⁰ T cells, between 10⁷ to 10⁹ T cells, or between 10⁸ to 10⁹ T cells. In some embodiments, the dose of T cells per subject is between 1×10⁶ to 1×10¹¹ cells, between 1×10⁶ to 5×10⁹ cells, between 1×10⁶ to 1×10⁸ cells, between 1×10⁶ to 5×10⁷ cells, between 1×10⁶ to 1×10⁷ cells, between 1×10⁶ to 5×10⁶ cells, between 5×10⁶ to 1×10¹¹, between 5×10⁶ to 1×10⁹, between 5×10⁶ to 5×10⁹, between 5×10⁶ to 1×10⁹, between 5×10⁶ to 5×10⁸, between 5×10⁶ to 1×10⁸, between 5×10⁶ to 5×10⁷, between 5×10⁶ to 1×10⁷, between 1×10⁷ to 1×10¹⁰ cells, between 1×10⁷ to 5×10⁹ cells, between 1×10⁷ to 5×10⁹ cells, between 1×10⁷ to 1×10⁹ cells, between 1×10⁷ to 5×10⁸ cells, between 1×10⁷ to 1×10⁸ cells, between 1×10⁷ to 5×10⁷ cells, between 5×10⁷ to 1×10¹⁰, between 5×10⁷ to 5×10⁹, between 5×10⁷ to 1×10⁹, between 5×10⁷ to 5×10⁸, between 5×10⁷ to 1×10⁸, between 1×10⁸ to 1×10⁹, between 1×10⁸ to 5×10⁸, between 5×10⁸ to 1×10⁹, between 5×10⁸ to 5×10⁹, between 5×10⁸ to 1×10⁹, between 1×10⁹ to 1×10¹⁰, or between 1×10⁹ to 5×10⁹ cells per patient.

In any of the treatment methods involving the use of the modified immune cells disclosed herein, the subject may be administered IL-2 concurrently with the cell therapy. More specifically, an effective amount of IL-2 may be given to the subject via a suitable route before, during, or after the cell therapy. In some embodiments, IL-2 is given to the subject after administration of the immune cells.

In some embodiments, prior to the cell therapy, the subject receives a lymphodepleting treatment to condition the subject for the cell therapy. In one embodiment, a lymphodepleting treatment regimen comprises cyclophosphamide 500 mg/m² intravenously and fludarabine 30 mg/m² intravenously is administered on the fifth, fourth, and third day before infusion of ICAM-1 CAR T cells, unless the combination is not tolerated. If it is determined that combination-based lymphodepletion presents an unfavorable safety risk to a subject, then lymphodepletion may proceed with cyclophosphamide or bendamustine alone.

### (B) Monitoring CAR-T Cell Distribution in a Patient

In another aspect, the present disclosure provides a method for monitoring CAR-T cell distribution in a patient undergoing CAR-T cell therapy. The method involves the use of CAR-T cells co-expressing human somatostatin receptor 2 (SSTR2) as a cell surface marker for monitoring CAR-T cell distribution in a patient.

In an embodiment, the method of cell therapy further comprises: (a) incubating a population of CAR-T cells described herein with a radioactive label that binds to SSTR2; (b) intravenously infusing the labeled CAR-T cells into a patient in an amount of 10⁷ to 10¹⁰ T cells/patient, and (c) detecting the labeled CAR-T cell distribution by positron emission tomography/computed tomography (PET/CT) imaging, wherein the labeled CAR-T cells are infiltrated into cancer cells to kill the cancer cells. In this method, SSTR2 is pre-labeled in vitro.

In another embodiment, the method comprises: (a) intravenously infusing a population of CAR-T cells described herein into a patient; (b) injecting into the patient a radioactive label that binds to SSTR2 at least one hour prior to PET/CT imaging, and (c) detecting the labeled CAR-T cell distribution by PET/CT imaging, wherein the labeled CAR-T cells are infiltrated into cancer cells to kill the cancer cells. In this method, SSTR2 is labeled post-infusion in vivo. In certain embodiments, the CAR-T cells have been transduced to express at least 100,000 molecules of SSTR2 per T cell.

SSTR2 can be used in conjunction with FDA-approved positron emission tomography (PET) radiotracers currently used in clinics to probe for overexpressed SSTR2 in neuroendocrine tumors, specifically ⁶⁸Gallium conjugates of DOTATOC and DOTATATE. Single-photon emission computed tomography (SPECT)-based imaging is also available using ¹¹¹In-DTPAOC (Octreoscan) or ¹⁷⁷Lutetium. SSTR2 displays restricted basal expression in tissues and all major organs except in the kidneys and cerebrum making it ideal for detection of adoptively transferred CAR-T cells targeting a multitude of solid tumors.

It has previously been shown that SSTR2 facilitates rapid radiotracer uptake and this combined with swift renal clearance of unbound DOTATOC means that high quality, clinical-grade images can be obtained at one hour post DOTATOC injection. DOTATOC also has a short half-life of 68 min which, combined with its rapid clearance, delivers a low radiation dose to the patient. The fact that SSTR2 is of human origin also limits its immunogenicity which has plagued experiments using non-human genetic reporters.

In some embodiments, the label is radioactively labeled DOTATOC or radioactively labeled DOTATATE, such as ⁶⁸Gallium-DOTATOC or ⁶⁸Gallium-DOTATATE. The methods for monitoring distribution of radiolabeled CAR-T cells may be used in connection with treating any of the carcinomas described herein, including but not limited to non-small cell lung carcinoma, small cell lung carcinoma, squamous cell lung carcinoma, large cell lung carcinoma, cervical carcinoma, hepatocellular carcinoma, renal carcinoma, bladder carcinoma, head and neck carcinoma, adenocarcinomas thereof, and squamous cell carcinomas thereof. SSTR2 can be used in conjunction with FDA-approved positron emission tomography (PET) radiotracers currently used in clinics to probe for overexpressed SSTR2 in neuroendocrine tumors, specifically ⁶⁸Gallium conjugates of DOTATOC and DOTATATE. Single-photon emission computed tomography (SPECT)-based imaging is also available using ¹¹¹In-DTPAOC (Octreoscan) or ¹⁷⁷Lutetium. SSTR2 displays restricted basal expression in tissues and all major organs except in the kidneys and cerebrum making it ideal for detection of adoptively transferred CAR-T cells targeting a variety of solid tumors.

In some embodiments, the labeled CAR-T cells are administered in an amount between 1×10⁶ to 1×10¹⁰ cells, between 1×10⁶ to 5×10⁹ cells, between 1×10⁶ to 1×10⁸ cells, between 1×10⁶ to 5×10⁷ cells, between 1×10⁶ to 1×10⁷ cells, between 1×10⁶ to 5×10⁶ cells, between 5×10⁶ to 1×10¹⁰, between 5×10⁶ to 1×10⁹, between 5×10⁶ to 5×10⁹, between 5×10⁶ to 1×10⁹, between 5×10⁶ to 5×10⁸, between 5×10⁶ to 1×10⁸, between 5×10⁶ to 5×10⁷, between 5×10⁶ to 1×10⁷, between 1×10⁷ to 1×10¹⁰ cells, between 1×10⁷ to 5×10⁹ cells, between 1×10⁷ to 5×10⁹ cells, between 1×10⁷ to 1×10⁹ cells, between 1×10⁷ to 5×10⁸ cells, between 1×10⁷ to 1×10⁸ cells, between 1×10⁷ to 5×10⁷ cells, between 5×10⁷ to 1×10¹⁰, between 5×10⁷ to 5×10⁹, between 5×10⁷ to 1×10⁹, between 5×10⁷ to 5×10⁸, between 5×10⁷ to 1×10⁸, between 1×10⁸ to 1×10⁹, between 1×10⁸ to 5×10⁸, between 5×10⁸ to 1×10⁹, between 5×10⁸ to 5×10⁹, between 5×10⁸ to 1×10⁹, between 1×10⁹ to 1×10¹⁰, or between 1×10⁹ to 5×10⁹ cells per patient.

SSTR2 compositions and methods for using SSTR2 as a reporter for CAR-T cell monitoring and use in cancer are disclosed in U.S. Pat. No. 10,577,408, which is expressly incorporated by reference herein.

### (C) Suicide Switch

SSTR2 can function as a dual reporter-suicide gene by conjugation of the therapeutic high-energy radioisotopes ¹⁷⁷Lutetium, ⁹⁰Yttrium, or ²¹³Bismuth to DOTATOC instead of ⁶⁸Gallium, thus enabling elimination of SSTR2 expressing T cells in the case of CAR toxicity. Further, because SSTR2 is surface expressed, it does not require prior radioligand internalization into the cell.

In some embodiments, the CAR-T cells express SSTR2, and the subject is administered a safety switch comprising an SSTR2-targeted cytotoxic conjugate if unacceptable toxicity from the CAR-T cells is identified, the conjugate comprising a cytotoxic agent coupled to an SSTR2 targeting moiety by a linker. In some embodiments, the targeting moiety is somatostatin, a somatostatin analog, octreotide, lanreotide, lutathera (¹⁷⁷Lu-DOTATATE), ⁹⁰Y-DOTATOC, Tyr3-octreotate (TATE), vapreotide, cyclo(AA-Tyr-DTrp-Lys-Thr-Phe) where AA is α-N-Me lysine or N-Me glutamic acid, pasireotide, lanreotide, seglitide, or an anti-SSTR2 antibody.

In some embodiments, the cytotoxic agent is a maytansinoid, a camptothecin, a dolastatin, an aurastatin, a pyrrolobenzodiazepine (PBD), a calicheamicin, a duocarmycin, a tubulolysin, an analogue thereof, or a combination thereof. In some embodiments, the cytotoxic agent is deruxtecan, exatecan, SN-38, monomethyl auristatin E (MMAE), monomethyl auristatin F (MMAE), Auristatin F-hydroxypropylamide, Aur0101, emtansine (DM1), Maytansinoid DM4, duocarmycin, duostatin-5, or a combinations thereof.

In some embodiments, the linker is a cleavable linker selected from the group consisting of maleimidocaproyl-L-valine-L-citrulline-p-aminobenzoyl carbamate (mc-VC-PABC) and maleimido-dPEG8-L-valine-L-alaline-p-aminobenzoyl carbamate (M-dPEG8-VA-PABC). In some embodiments, the linker is a non-cleavable linker selected from the group consisting of maleimidocaproyl (MC), succinimidyl-4-(N-maleimidomethyl)cyclohexane-1-carboxylate (SMCC), and thioether-containing linker.

In some embodiments, the cytotoxic conjugate has a drug-to-antibody (DAR) ratio between 2-10. In some embodiments, the cytotoxic conjugate is PEN-221.

The details of one or more embodiments of the invention are set forth in the description below. Other features or advantages of the present invention will be apparent from the following drawings and detailed description of several embodiments, and also from the appended claims.

### (D) Co-Administration of Tyrosine Kinase Inhibitors

It is known that transient inhibition or modulation of TCR signaling and/or CAR signaling in human T cells can prevent or reverse T cell exhaustion and restore T cell function to CAR-T cells undergoing functional exhaustion. In particular, in vivo treatment of the CAR-T cells described herein with certain tyrosine kinase inhibitors inhibiting T cell receptor signaling (e.g., a Lck tyrosine kinase inhibitor (e.g., dasatinib)) can suppress exhaustion marker expression, augment memory formation, decrease expression of PD1 on CAR-T cells, and facilitate cell survival/proliferation as described in US 2020/101108 A1 and US 2021169880 A1, the disclosures of which are expressly incorporated by reference herein. Similar findings have been obtained using small molecules having a thiazole, imidazolepyridiazine or piperazinyl-methyl-aniline structure (US 2021/0393628 A1, incorporated by reference herein).

Thus, in some embodiments, a patient receiving CAR-T treatment in accordance with the present disclosure may be additionally administered a tyrosine kinase or small molecule having a thiazole, imidazolepyridiazine or piperazinyl-methyl-aniline structure to mitigate T cell exhaustion, augment memory T cell formation, and/or maintain and facilitate cell survival and proliferation. In other embodiments, these active agents may be used to improve ex vivo cell expansion and collection of CAR-T populations that are resistant and/or less prone to T cell exhaustion. Thus, in another aspect, the present disclosure further provides cell compositions comprising a population of CAR-T cells that are expanded in the presence of particular compounds described herein.

Exemplary tyrosine kinases for administration or cell treatment include dasatinib, ponatinib, saracatinib, bosutinib, nilotinib, and combinations thereof. Exemplary small molecules having a thiazole, imidazolepyridiazine or piperazinyl-methyl-aniline structure are disclosed in US 2021/0393628 A1, the disclosures of which are expressly incorporated by reference herein.

Such methods are not limited to particular manner of administration. In some embodiments, multiple cycles of treatment are administered to the subject. In some embodiments, the pharmaceutical composition is administered intermittently. In some embodiments, the pharmaceutical composition is administered for a period of time sufficient to restore at least partial T cell function then discontinued. In some embodiments, the pharmaceutical composition is administered orally.

In some embodiments, the pharmaceutical compositions are administered iteratively for purposes of facilitating periods of CAR-T cell inactivation (e.g., during pharmaceutical composition administration) and periods of CAR-T cell activation (e.g., during absence of pharmaceutical composition administration; following clearance of the pharmaceutical composition).

In some embodiments, patients undergoing CAR-T treatment are subjected to intermittent exposure to dasatinib (or other active agents above) to reduce exhaustion, and augment the engraftment, proliferation, and persistence of CAR-T cells in vivo., as well as antitumor function of the CAR-T cells.

The terms “intermittent administration” or “administered intermittently” in connection with the tyrosine kinase inhibitors described herein refer to the use of these tyrosine kinase inhibitors in an administration regime that causes intermittent changes between a state wherein the patient has tyrosine kinase inhibitor serum levels within the therapeutic window and a state wherein the patient has tyrosine kinase inhibitor serum levels below the therapeutic window. A therapeutic window of a given tyrosine kinase inhibitor can be determined by any methods known in the art.

Alternatively, the terms “intermittent administration” and “administered intermittently” in connection with a tyrosine kinase inhibitor as used herein refer to the use of a tyrosine kinase inhibitor in an administration regime causing: (1) intermittent changes between a state where the patient has tyrosine kinase inhibitor serum levels causing complete inhibition of the tyrosine kinase and a state where the patient has tyrosine kinase inhibitor serum levels causing partial inhibition of the tyrosine kinase; (2) intermittent changes between a state where the patient has tyrosine kinase inhibitor serum levels causing complete inhibition of the tyrosine kinase and a state where the patient has tyrosine kinase inhibitor serum levels causing no inhibition of the tyrosine kinase; or (3) intermittent changes between a state where the patient has tyrosine kinase inhibitor serum levels causing partial inhibition of the tyrosine kinase and a state where the patient has tyrosine kinase inhibitor serum levels causing no inhibition of the tyrosine kinase.

Such inhibition can be measured by any methods known in the art, e.g., by measuring the activity of the tyrosine kinase itself using appropriate enzyme assays, or by measuring cellular functions downstream of the kinase. In some embodiments, a partial inhibition refers to an inhibition of at least 25% to 75% compared to a situation in the absence of the inhibitor. As used herein, “no inhibition” refers to an inhibition of less than 25%, or less than 10% compared to a situation in the absence of the inhibitor.

In the case of CAR-T cells, inhibition of less than 25% or 10% can be an inhibition of the cytotoxic lysis, cytokine secretion, and/or proliferation of the T cells. Further, the inhibition of at least 25%, but no more than 75%, can preferably be an inhibition of the cytotoxic lysis, cytokine secretion, and proliferation of the CAR-T cells.

In some embodiments, intermittent administration of dasatinib may cause intermittent changes between a state wherein the serum levels of dasatinib are above 50 nM and a state wherein the serum levels of dasatinib are at or below 50 nM. Intermittent administration may be achieved by using an administration interval longer than the terminal phase half-life of the tyrosine kinase inhibitor, longer than 2 times the terminal phase half-life of the tyrosine kinase inhibitor, or longer than 3 times, 4 times, or 5 times the terminal phase half-life of the tyrosine kinase inhibitor. For example, intermittent administration of dasatinib may be achieved using an administration interval of at least 6 hours for dasatinib or at least 12 hours for dasatinib. It will be understood by a person skilled in the art that for each administration regime, appropriate dosages of the respective tyrosine kinase inhibitors can be selected based on pharmacokinetic and pharmacodynamic experiments.

The terms “continuous administration” or “administered continuously” in connection with a tyrosine kinase inhibitor as used herein refer to the use of said tyrosine kinase inhibitor in an administration regime that causes a complete inhibition of the tyrosine kinase in a continuous manner. According to the invention, a complete inhibition refers to an inhibition of at least 75%, compared to a situation in the absence of the inhibitor. Such inhibition can be measured by any methods known in the art, e.g., by measuring the activity of the tyrosine kinase itself using appropriate enzyme assays, or by measuring cellular functions downstream of said kinase.

In the case of CAR-T cells, inhibition of at least 75% can be an inhibition of the cytotoxic lysis, cytokine secretion, and proliferation of T cells. Alternatively, the terms “continuous administration” and “administered continuously” in connection with a tyrosine kinase inhibitor described herein refer to use of the tyrosine kinase inhibitor in an administration regime that results in serum levels of the tyrosine kinase which are continuously within the therapeutic window. In some embodiments, continuous administration of dasatinib encompasses any administration wherein the serum levels of dasatinib are constantly maintained at or above 50 nM. In one embodiment, dasatinib is administered continuously, where the administration comprises oral administration of 50-200 mg dasatinib every 6-8 hours or 140 mg every 6 hours.

In some embodiments, the threshold serum level is within the range of 0.1 nM-1 μM, 1 nM-500 nM, 5 nM-100 nM, 10 nM-75 nM, or 25 nM-50 nM.

### (E) Combination Therapies

The immune cell populations comprising e.g., the CAR-T cells described herein may be utilized in conjunction with other types of therapy or active agents for cancer, such as chemotherapy, surgery, radiation, gene therapy, and so forth. Such therapies can be administered simultaneously or sequentially (in any order) with the immunotherapy described herein. When co-administered with an additional therapeutic agent, suitable therapeutically effective dosages for each agent may be lowered due to additive or synergistic effects.

In some examples, the subject is treated with an anti-cancer therapy (e.g., those disclosed herein) to reduce tumor burden prior to the CAR-T therapy disclosed herein. For example, the subject (e.g., a human cancer patient) may be treated with chemotherapy (e.g., comprising a single chemotherapeutic agent or a combination of two or more chemotherapeutic agents) at a dose that substantially reduces tumor burden. In some instances, the chemotherapy may reduce the total white blood cell count in the subject to lower than 10⁸/L, e.g., lower than 10⁷/L. Tumor burden of a patient after the initial anti-cancer therapy, and/or after the CAR-T cell therapy disclosed herein may be monitored via routine methods. If a patient showed a high growth rate of cancer cells after the initial anti-cancer therapy and/or after the CAR-T therapy, the patient may be subjected to a new round of chemotherapy to reduce tumor burden followed by any of the CAR-T therapies disclosed herein.

Non-limiting examples of other anti-cancer therapeutic agents for use in combination with the modified immune cells (e.g., CAR-T cells) described herein include, but are not limited to, immune checkpoint inhibitors (e.g., PDL1, PD1, and CTLA4 inhibitors), anti-angiogenic agents (e.g., TNP-470, platelet factor 4, thrombospondin-1, tissue inhibitors of metalloproteases, prolactin, angiostatin, endostatin, bFGF soluble receptor, transforming growth factor beta, interferon alpha, interferon gamma, soluble KDR and FLT-1 receptors, and placental proliferin-related protein); VEGF antagonists (e.g., anti-VEGF antibodies, VEGF variants, soluble VEGF receptor fragments); chemotherapeutic compounds. In some embodiments, the anti-cancer therapeutic agents include pembrolizumab (Keytruda™), ipilimumab (Yervoy™), nivolumab (Opdivo™), or atezolizumab (Tecentriq™). Exemplary chemotherapeutic compounds include pyrimidine analogs (e.g., 5-fluorouracil, floxuridine, capecitabine, gemcitabine and cytarabine); purine analogs (e.g., fludarabine); folate antagonists (e.g., mercaptopurine and thioguanine); antiproliferative or antimitotic agents, for example, vinca alkaloids; microtubule disruptors such as taxane (e.g., paclitaxel, docetaxel), vincristin, vinblastin, nocodazole, epothilones and navelbine, and epidipodophyllotoxins; and DNA damaging agents (e.g., actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethyhnelamineoxaliplatin, iphosphamide, melphalan, mechlorethamine, mitomycin, mitoxantrone, nitrosourea, plicamycin, procarbazine, taxol, taxotere, teniposide, triethylenethiophosphoramide and etoposide).

In another embodiment, the CAR-T cells co-express an anti-inflammatory cytokine or are co-administered with an anti-inflammatory cytokine, such as IL-4, IL-10, IL-11, IL-13, or a combination thereof.

In another embodiment, the CAR-T co-express or are co-administered with a proinflammatory cytokine antagonist targeting or inhibiting IFNγ, IL1α, IL1β, IL-1, IL-6, IL-12, GM-CSF, or a combination thereof. In some embodiments, the proinflammatory cytokine antagonist targets or inhibits a cytokine selected from the group consisting of IL-2, IL-5, IL-7, IL-8, IL-9, IL-15, IL-17, IL-18, IL-21, IL-23, sIL-1RI, sIL-2Rα, sIL-6R, IFNα, IFNβ, MIPα, MIPβ, CSF1, LIF, G-CSF, CXCL10, CCL5, eotaxin, TNF, MCP1, MIG, RAGE, CRP, angiopoietin-2, VWF, TGFα, VEGF, EGF, HGF, FGF, perforin, granzyme, and ferritin.

In another embodiment, one or more therapeutic antibodies are administered in combination with the CAR-T cells. In an embodiment, the therapeutic antibody is selected from the group consisting of abagovomab, adecatumumab, afutuzumab, alemtuzumab, altumomab, amatuximab, anatumomab, arcitumomab, bavituximab, bectumomab, bevacizumab, bivatuzumab, blinatumomab, brentuximab, cantuzumab, catumaxomab, cetuximab, citatuzumab, cixutumumab, clivatuzumab, conatumumab, daratumumab, drozitumab, duligotumab, dusigitumab, detumomab, dacetuzumab, dalotuzumab, ecromeximab, elotuzumab, ensituximab, ertumaxomab, etaracizumab, farietuzumab, ficlatuzumab, figitumumab, flanvotumab, futuximab, ganitumab, gemtuzumab, girentuximab, glembatumumab, ibritumomab, igovomab, imgatuzumab, indatuximab, inotuzumab, intetumumab, ipilimumab, iratumumab, labetuzumab, lexatumumab, lintuzumab, lorvotuzumab, lucatumumab, mapatumumab, matuzumab, milatuzumab, minretumomab, mitumomab, moxetumomab, namatumab, naptumomab, necitumumab, nimotuzumab, nofetumomab, ocaratuzumab, ofatumumab, obinutuzumab, olaratumab, onartuzumab, oportuzumab, oregovomab, panitumumab, parsatuzumab, patritumab, pemtumomab, pertuzumab, pintumomab, pritumumab, racotumomab, radretumab, rilotumumab, rituximab, robatumumab, satumomab, sibrotuzumab, siltuximab, simtuzumab, solitomab, tacatuzumab, taplitumomab, tenatumomab, teprotumumab, tigatuzumab, tositumomab, trastuzumab, tucotuzumab, ublituximab, veltuzumab, vorsetuzumab, votumumab, zalutumumab, CC49, and combinations thereof.

In some embodiments, radiation or radiation and chemotherapy is used in combination with the cell populations comprising modified immune cells described herein. Additional useful agents and therapies can be found in Physician's Desk Reference, 59th edition, (2005), Thomson P D R, Montvale N.J.; Gennaro et al., Eds. Remington's The Science and Practice of Pharmacy 20th edition, (2000), Lippincott Williams and Wilkins, Baltimore Md.; Braunwald et al., Eds. Harrison's Principles of Internal Medicine, 15th edition, (2001), McGraw Hill, NY; Berkow et al., Eds. The Merck Manual of Diagnosis and Therapy, (1992), Merck Research Laboratories, Rahway N.J.

### III. Genetically Modified Immune Cells

In another aspect, the present disclosure provides a population of genetically modified immune cells (e.g., T cells) expressing a chimeric antigen receptor (CAR) construct disclosed herein. Such modified immune cells express a CAR which specifically binds ICAM-1, thereby eliminating the target disease cells via, e.g., the effector activity of the immune cells. In some embodiments, the population of immune cells comprises genetically modified cytotoxic effector cells (CAR-T cells) independently expressing a single CAR, a bispecific or multispecific CAR, or multiple CARs (e.g., dual CAR-T).

In some embodiments, the immune cells for gene transduction herein may be T cells. In other embodiments, the immune cells for gene transduction are natural killer (NK) cells. In other embodiments, the immune cells for gene transduction are tumor infiltrating lymphocytes, dendritic cells, monocytes, macrophages, B cells, neutrophils, eosinophils, basophils, mast cells, myeloid-derived suppressor cells, mesenchymal stem cells, precursors thereof, subtypes thereof, or combinations thereof.

T cells may be selected from the group consisting of cytotoxic T-lymphocytes (CD8+), including Tc1, Tc2, Tc9, Tc17, and Tc22 T cells; helper T-lymphocytes (CD4+), including Th1, Th2, Th17, Th9, and Tfh T cells; antigen-inexperienced naïve T cells (T_{N}), stem cell memory T cells (Tscm or T_{SCM}), central memory T cells (Tcm or T_{CM}), effector memory T cells (Tem or T_{EM}), effector T cells (Teff, T_{EFF} or T_{E}), precursors to an exhausted T cell (Tpex or T_{PEX}), or exhausted T cells (Tex or T_{EX}), central memory T cells, effector memory T cells, tissue resident memory T cells, virtual memory T cells, natural killer T cells (NKT cells), FOXP3+ T cells, FOXP3− T cells). T cells may be purified from peripheral blood lymphocytes by methods known to those skilled in the art. In some embodiments, T cells may be cultured, expanded, differentiated or de-differentiated to obtain particular T cell subsets prior to or following transduction, such as antigen-inexperienced naïve T cells (T_{N}), stem cell memory T cells (Tscm or T_{SCM}), and/or central memory T cells (Tcm or T_{CM}).

In some embodiments, the immune cells are stem cells or are derived from stem cells. The stem cells can be adult stem cells, non-human embryonic stem cells, more particularly non-human stem cells, mesenchymal stem cells, cord blood stem cells, progenitor cells, bone marrow stem cells, induced pluripotent stem cells, totipotent stem cells or hematopoietic stem cells. Representative human cells are CD34+ cells. In other embodiments, the immune cell is derived from the differentiation of a population of induced pluripotent cells (iPSCs).

In some embodiments, the immune cells are harvested directly from a subject, e.g., a human subject. The cells are genetically modified as described herein and the genetically engineered immune cells are infused back into the same subject, for example, in a CAR-T cell therapy. In this case, the genetically engineered immune cells are autologous to the subject receiving the CAR-T cell therapy. In another embodiment, the immune cells are harvested directly from a donor subject, modified, and the genetically engineered immune cells are infused into a recipient subject in need of therapy, e.g., a CAR-T cell therapy. In this case, the donor immune cells are HLA-matched to the recipient subject, i.e., the cells are allogeneic to the recipient subject. In some embodiments, the immune cells are harvested and isolated from the peripheral blood of the subject (e.g., peripheral blood lymphocytes) and expanded in vitro prior to the genetic modifications disclosed herein.

In some embodiments, the CAR-expressing immune cells, including CAR-T cells, are transduced to additionally express one or more gene products, including, but not limited to SSTR2, immune checkpoint inhibitors, cytokines, and the like.

In some embodiments, immune effector cells can be genetically modified with one or more CARs recognizing target cells using combinatorial Boolean logic: one can engineer T cells with multi-receptor circuits that function as AND gates (requiring two antigens to be present), OR gates (requiring the presence of one of two possible antigens), and NOT gates (high expression of one antigen, low expression of another) to increase tumor selectivity by limiting cross-reactivity with healthy tissues that also express the CAR/TCR target antigen. Exemplary target antigen combinations for AND and AND-NOT logic gates are disclosed in Table 1 of WO 2022/036133 where an “AND” precedes or follows a target antigen present on the surface of a target cancer cell and a “NOT” precedes an antigen that that is not present on the surface of a target cancer cell, but may be present on the surface of a non-cancerous cell.

Where a target antigen pair (or triple) provides for an AND logic gate, two (or three) antigens must be present on the surface of a target cancer cell in order for a genetically modified cytotoxic immune cell of the present disclosure to kill the target cancer cell, where in this case the genetically modified cytotoxic immune cell is genetically modified to express two or three antigen-triggered polypeptides, each recognizing one of the target antigens of the target antigen pair/triplet. For example, where a target antigen pair provides an AND gate logic, each of the target antigens of the target antigen pair must be present on the surface of a target cancer cell in order for a genetically modified cytotoxic immune cell of the present disclosure to kill the target cancer cell.

Where a target antigen pair/triple provides an AND-NOT logic gate (or, correspondingly, a NOT-AND logic gate), a genetically modified cytotoxic immune cell of the present disclosure: a) is activated to kill a target cancer cell that expresses the AND target cell surface antigen (e.g., the first target cell surface antigen), but not the NOT target cell surface antigen (e.g., the second and/or third target cell surface antigen), on its cell surface; and b) is inhibited from killing a non-cancerous cell if the non-cancerous cell expresses both the AND target cell surface antigen and the NOT target cell surface antigen(s) on its cell surface. In these cases, the genetically modified cytotoxic immune cell must express at least a first antigen-triggered polypeptide that specifically binds the AND target antigen of the target antigen pair and a second triggered polypeptide that specifically binds the NOT antigen of the target antigen pair. For example, in some cases, binding of an antigen-triggered polypeptide to the NOT target cell surface antigen (expressed on a non-cancerous cell) inhibits T cell activation. In this manner, unintended/undesired killing of a non-cancerous cell is reduced, because the target cancer cell expressing the AND target antigen and not the NOT target antigen will be preferentially killed over the non-cancerous cell expressing both the AND target antigen and the NOT target antigen. Since the cancer cell does not express the NOT target cell surface antigen (expressed on a non-cancerous cell), binding of the first antigen-triggered polypeptide to the AND target antigen (present on the cancer cell surface) results in activation of the genetically modified cytotoxic T cell and killing of the cancer cell.

In some embodiments, the population of immune cells comprising the CAR-expressing cells further includes a second population of immune cells. In some embodiments, the second population of immune cells includes non-transduced immune cells, immune cells expressing another CAR, and/or immune cells expressing another gene product.

In some embodiments, the population of immune cells comprising the CAR disclosed herein may comprise at least about 10%, 20%, 30%, 40%, 50%, 60%, 70%, 80%, 90%, or more of the total immune cell population, or a range between two of the foregoing amounts. In one embodiment, about 50-70% of the immune cells may express the CAR.

### IV. Methods of Preparing Modified Immune Cells

The ICAM-1 CAR and optionally other exogenous nucleic acids can be introduced into suitable immune cells by routine methods and/or approaches. To generate modified immune cells expressing the ICAM-1-directed CARs described herein, coding sequences from one or more CARs, and optionally other gene products, may be cloned into a suitable expression vector (e.g., including but not limited to lentiviral vectors, retroviral vectors, adenoviral vectors, adeno-associated vectors, PiggyBac transposon vector and Sleeping Beauty transposon vector) and introduced into host immune cells using conventional recombinant technology known to those skilled in the art. As a result, modified immune cells of the present disclosure may comprise one or more exogenous nucleic acids encoding at least one CAR and optionally one or more other gene products described herein. In some instances, the coding sequences of such molecules are integrated into the genome of the cell for expression using viral expression vectors (e.g., lentivirus vectors) or by gene editing into suitable target sites. In other instances, the coding sequences of such molecules are not integrated into the genome of the cell.

In an embodiment, the expression vector for transducing the ICAM-1 CAR and/or other exogenous gene products may be a lentivirus. In one embodiment, the expression vector or lentivirus comprises an ICAM-1 CAR coding region comprising from 5′ to 3′, an F292A mutant I domain of LFA-1, a CD8 hinge region, a CD28 transmembrane domain, a CD28 co-stimulatory domain, a 4-1BB co-stimulatory domain, and a CD3ζ signaling domain. In an exemplary embodiment, the expression vector or lentivirus encoding an ICAM-1 CAR according to the present disclosure comprises a nucleic acid sequence set forth in SEQ ID NO: 25 or an amino acid sequence set forth in SEQ ID NOs: 27.

In another embodiment, the expression vector or lentivirus encodes an ICAM-1 CAR-SSTR2 fusion protein comprising from 5′ to 3′, a CD8 alpha signal peptide, a c-myc tag, an F292A mutant I domain of LFA-1, a CD8 hinge region, a CD28 transmembrane domain, a CD28 co-stimulatory domain, a 4-1BB co-stimulatory domain, and a CD3ζ signaling domain, a P2A element, and an SSTR2 polypeptide. In an exemplary embodiment, the expression vector or lentivirus encodes an ICAM-1 CAR-SSTR2 fusion protein comprising a nucleic acid sequence set forth in SEQ ID NO: 26 or an amino acid sequence set forth in any one of SEQ ID NOs: 28-31.

An exogenous nucleic acid comprising a coding sequence of interest may further comprise a suitable promoter, which can be in operable linkage to the coding sequence. A promoter, as used herein, refers to a nucleotide sequence in a nucleic acid to which RNA polymerase can bind to initiate the transcription of a DNA coding region into mRNA, which will then be translated into the corresponding protein. A promoter is considered to be “operably linked” to a coding sequence when it is in a correct functional location and orientation relative to the coding sequence to control (“drive”) transcriptional initiation to produce the mRNA for translating the protein. In some instances, the promoter described herein can be constitutive, which initiates transcription independent other regulatory factors. In some instances, the promoter described herein can be inducible, which is dependent on regulatory factors for transcription.

Additionally, the exogenous nucleic acids described herein may further contain, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColE1 for proper episomal replication; versatile multiple cloning sites; and T7 and SP6 RNA promoters for in vitro transcription of sense and antisense RNA.

In some instances, one or more nucleic acids encoding the CAR(s) and/or other gene products disclosed herein can be inserted into a suitable expression cassette in a multi-cistronic manner such that the various molecules are expressed as separate polypeptides. In some examples, an internal ribosome entry site (IRES) can be inserted between two coding sequences to achieve this goal. Alternatively, a nucleotide sequence coding for a self-cleaving peptide (e.g., T2A or P2A) can be inserted between two coding sequences as described above.

For example, in some embodiments, T cells may be transduced with a nucleic acid encoding a second gene product, such as SSTR2, and a CAR disclosed herein. In certain embodiments, the nucleic acid encodes an SSTR2 polypeptide (aa 1-381; SEQ ID NO: 16) or a truncated SSTR2 polypeptide (aa 1-314; SEQ ID NO: 17). In a specific embodiment, the nucleic acid encodes a SSTR2-CAR fusion protein comprising an amino acid cleavage sequence between the CAR coding sequence and the SSTR2 coding sequence. The cleavage sequence may encode a self-cleaving 2A peptide from porcine teschovirus-1 (P2A; SEQ ID NO: 21), equine rhinitis A virus (E2A; SEQ ID NO: 22), thosea asigna virus (T2A; SEQ ID NO: 23), foot-and-mouth disease virus (F2A; SEQ ID NO: 24), or a combination thereof.

### V. General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane, Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985; Transcription and Translation (B. D. Hames & S. J. Higgins, eds. (1984; Animal Cell Culture (R. I. Freshney, ed. (1986; Immobilized Cells and Enzymes (IRL Press, (1986); B. Perbal, A practical Guide To Molecular Cloning (1984); F. M. Ausubel et al. (eds.); Chimeric Antigen Receptor (CAR) Immunotherapy (D. W. Lee and N. N. Shah, eds., Elsevier, 2019, ISBN: 9780323661812); Basics of Chimeric Antigen Receptor (CAR) Immunotherapy (M. Y. Balkhi, Academic Press, Elsevier Science, 2019, ISBN: 9780128197479); Chimeric Antigen Receptor T Cells Development and Production (V. Picanço-Castro, K. C. R. Malmegrim, K. Swiech, eds., Springer US, 2020, ISBN: 9781071601488); Cell and Gene Therapies (C. Bollard, S. A. Abutalib, M.-A. Perales eds., Springer International, 2018; ISBN: 9783319543680) and Developing Costimulatory Molecules for Immunotherapy of Diseases (M. A. Mir, Elsevier Science, 2015, ISBN: 9780128026755).

The present disclosure is not limited in its application to the details of construction and the arrangements of component set forth in the description herein or illustrated in the drawings. The present disclosure is capable of other embodiments and of being practice or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items. As also used in this specification and the appended claims, the singular forms “a,” “an,” and “the” include plural references unless the context clearly dictates otherwise.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1

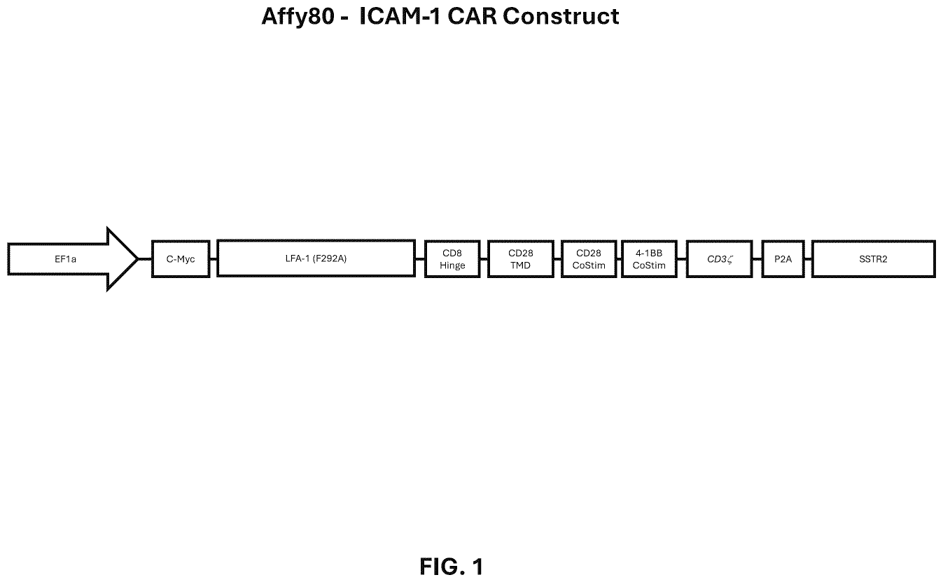

CAR-T cell manufacturing. Affy80 CAR-T cells were manufactured utilizing a lentivirus manufactured by Lentigen Corporation (now part of Miltenyi Biotec, Gaithersburg, MD). The Affy80-CAR-T cells were transduced with a lentivirus encoding an ICAM-1 CAR fusion protein comprised of (from 5′-3′) an EF1-alpha promoter, c-Myc epitope tag, a mutant I domain of LFA-1, a CD8 hinge region, a CD28 transmembrane domain, a CD28 co-stimulatory domain, a 4-1BB co-stimulatory domain, a CD3ζ signaling domain, a P2A self-cleaving peptide, and an SSTR2 coding region. The resulting construct (lentivirus not shown), Affy80, is depicted in FIG. 1. Affy80 comprises the nucleic acid sequence set forth in SEQ ID NO: 26, which encodes an amino acid sequence set forth in SEQ ID NO: 29.

Healthy donor leukopak cells were sorted using CD4 and CD8 microbeads and column. T cells were then activated with ImmunoCult CD3 and CD28 T cell activator and expanded in a G-Rex 6M plate for ten days. Twenty-four (24) hours post addition of cell activator, a recombinant CAR-expressing lentivirus was added to the well. Forty-eight (48) hours following addition of the virus, additional media is added, and cells are left to expand for 10 days.

In vitro target cell killing assay. 5×10⁴ target cells stably transduced to express GFP and firefly luciferase were co-cultured with ICAM-1 CAR-T cells at an effector to target ratio (E:T) of 5:1. Co-cultures were carried out in T cell culture medium containing 150 μg/mL D-Luciferin (Fisher Scientific, NC1276267) with no cytokine supplementation. Luminescence was measured using a plate reader (BioTek, Synergy Neo2) with readings every 24 hrs for 3 days. In each condition readings were normalized to the no T cell control group and percent cytotoxicity was reported.

Alternatively, 5×10⁴ primary healthy cells were co-cultured with c-Met CART cells (positive control), low affinity ICAM-1 CAR-T cells (Affy 80), and non-specific CAR-T cells (negative control) at an E:T ratio of 5:1. Co-cultures were carried out in T cell culture medium with no cytokine supplementation. Cytotoxicity was measured using label-free cellular impedance to continuously monitor and record cell killing (Agilent, xCELLigence Real-Time Cell Analysis (RTCA) system).

In vivo studies. An in vivo efficacy study was conducted to screen the potential efficacy of Affy80 in vivo. Cell lines were tested to confirm ICAM-1 expression prior to use in generating each in vivo model. NSCLC and CC models were generated by inoculation with NSCLC or CC tumor cells per indication via tail vein injection in NSG mice and weekly monitored by imaging to confirm establishment of tumors. All cell lines were in growth phase at the start of study to minimize model variability between mice. Mice were inoculated with 1.0×10⁷ NSCLC (H226, H441) or CC (SW756, HeLa) tumor cells. Three to five days post inoculation and upon confirmation of tumor growth, animals were randomized (Day 0) and dosed with either vehicle (PBS; n=5) or 6.5×10⁶ ICAM-1 CAR-T (Affy80) cells (n=5) (D1). Tumors were tracked by luciferase whole body imaging weekly. Initially, the imaging was conducted to visualize tumor size and randomize groups, followed by weekly imaging to monitor tumor size. Imaging was done using IVIS imaging technology. Mice were dosed with luciferin by intraperitoneal injection at 150 mg/kg in a 5 mL/kg volume. Animals were anesthetized via isoflurane inhalant and imaged 10 minutes post-luciferin dosing. Body weights & clinical observations were made twice per week. Imaging results are shown for primary tumor development prior to their appearance at secondary tumor sites.

### Example 2: ICAM-1 Expression in Human Lung Carcinoma Tissue Arrays

Two human lung carcinoma tissue arrays were evaluated for ICAM-1 expression using an immunohistochemistry kit according to manufacturer's instructions (Ultra Streptavidin HRP Kit (cat. #929501, multi-antibody species, DAB chromogen), Biolegend, San Diego, CA) using CD54/ICAM-1 (E3Q9N) XP® Rabbit mAb (Cell Signaling Technology, cat. #67836).

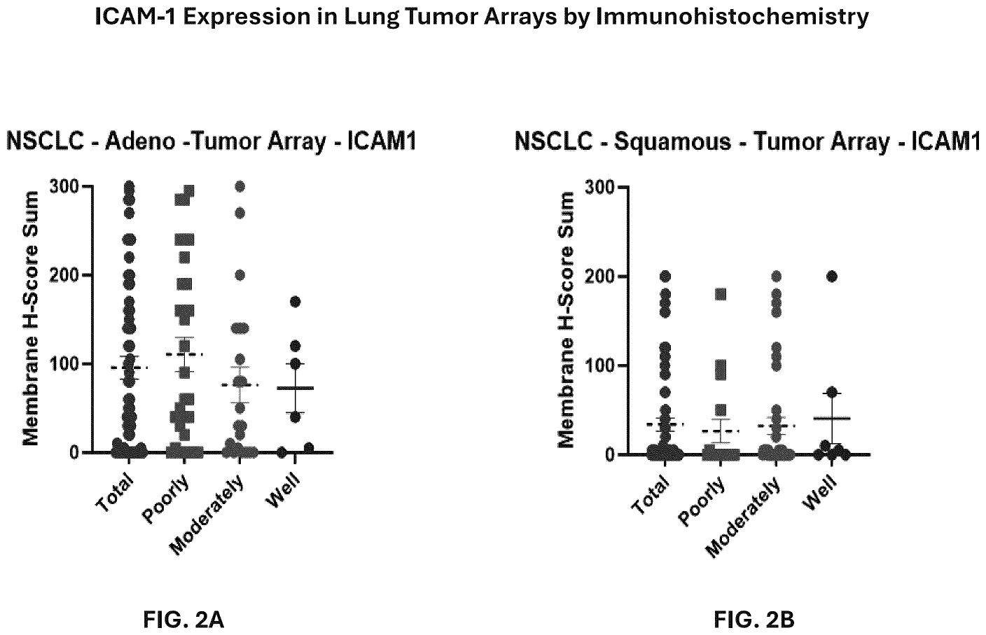

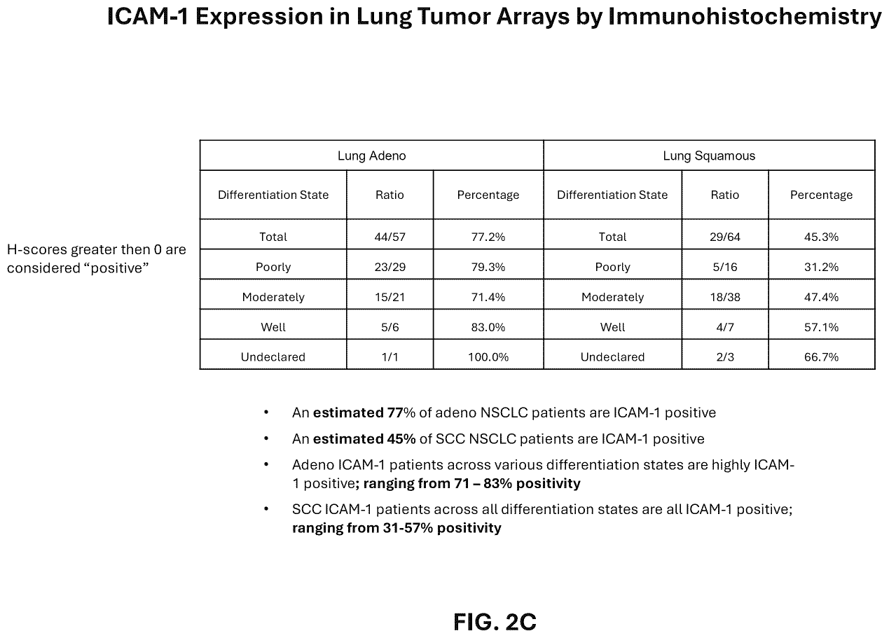

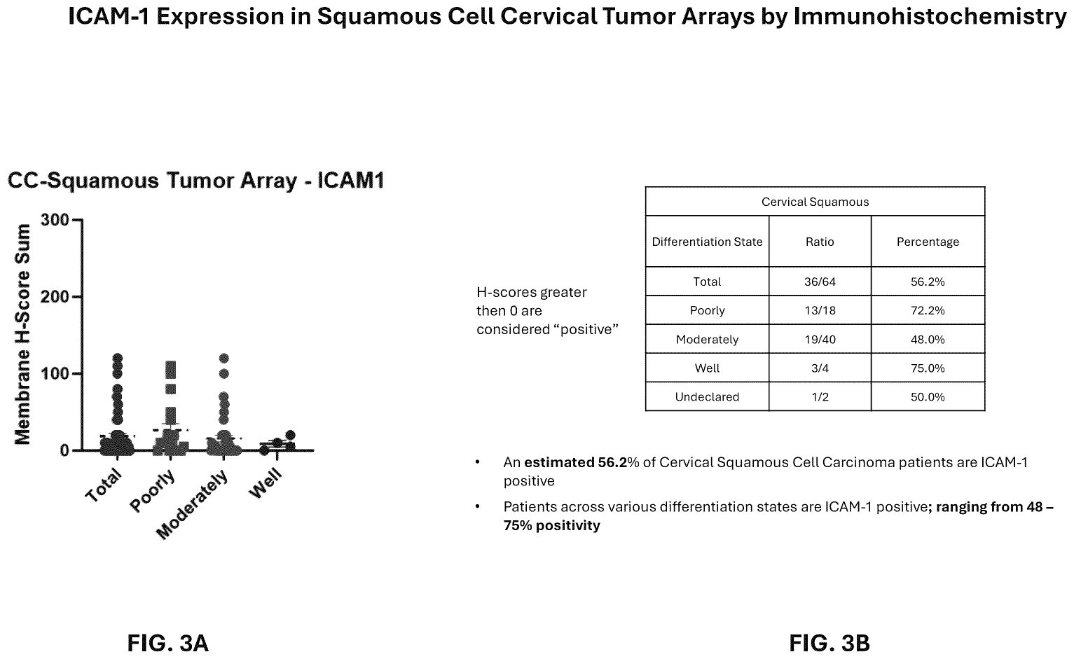

FIGS. 2-3 show ICAM-1 expression levels detected by immunohistochemistry in a human non-small cell lung carcinoma tissue array (Creative Bioarray, cat #LUTMA219) (FIG. 2A); a human non-small cell lung squamous carcinoma tissue array (Biochain, cat #T8235724) (FIG. 2B); and in a squamous cell cervical tumor array (Creative Bioarray, cat #UCTMA065) (FIG. 3).

### Example 3: In Vitro Cytotoxicity Assays Using Human Lung Carcinoma Cell Lines Co-Cultured with ICAM-1 CAR-T Cells

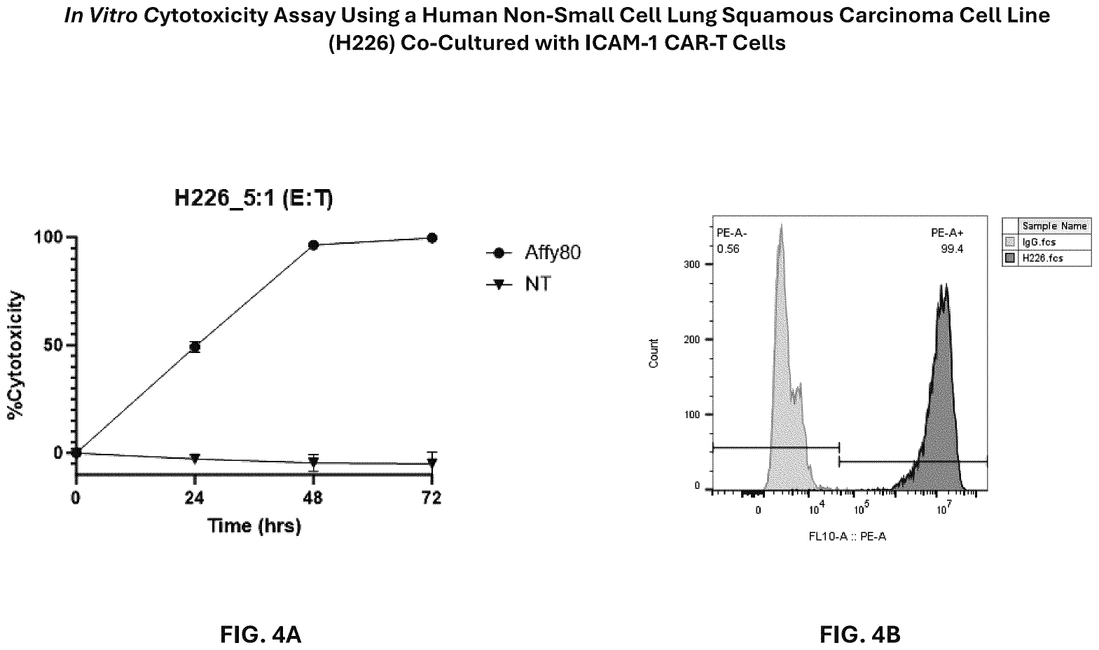

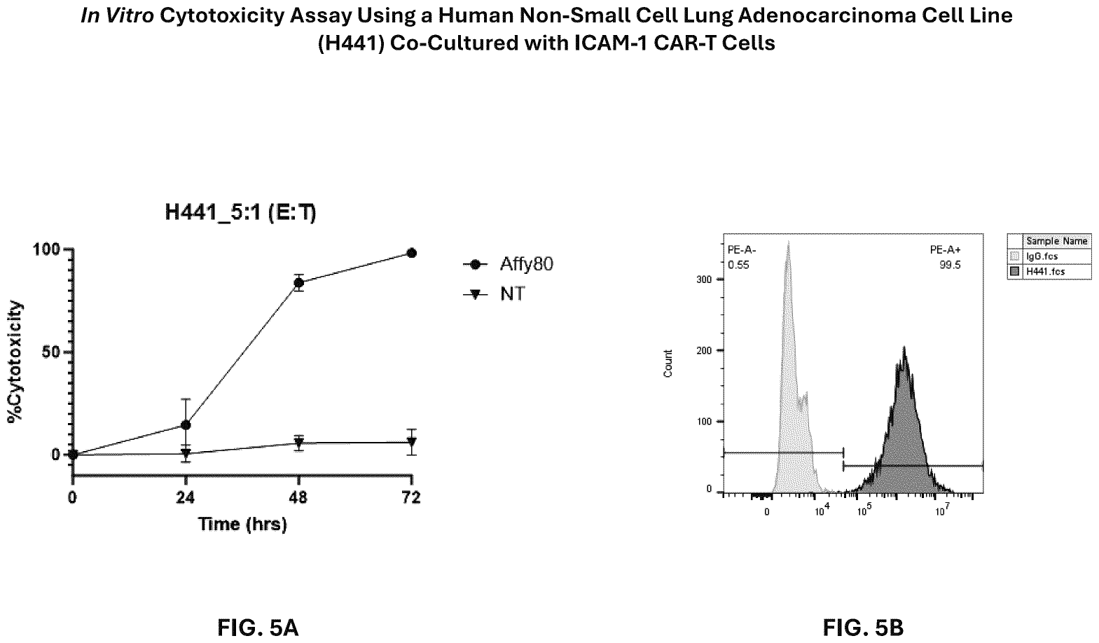

FIGS. 4-5 show the results from in vitro cytotoxicity assays using human lung carcinoma cell lines co-cultured with ICAM-1 CAR-T cells at an effector:target (E:T) ratio of 5:1. The ICAM-1 CAR-T cells were produced by transfection with Affy80, the recombinant lentivirus described in Example 1 and depicted in FIG. 1. The control cells were untransfected T cells (NT).

FIG. 4A is a graph showing % cytotoxicity as a function of time using a human non-small cell lung squamous carcinoma cell line (H226) co-cultured with ICAM-1 CAR-T cells. FIG. 4B is a histogram showing expression of ICAM-1 in H226 cells by flow cytometry following surface staining with an anti-ICAM-1 phycoerythrin (PE-A) conjugate (right peak) or staining with anti-IgG1 isotype control conjugated to phycoerythrin (PE-A-).

FIG. 5A is a graph showing % cytotoxicity as a function of time using a human non-small cell lung adenocarcinoma cell line (H441) co-cultured with ICAM-1 CAR-T cells. FIG. 5B is a histogram showing expression of ICAM-1 in H441 cells by flow cytometry following surface staining with an anti-ICAM-1 phycoerythrin (PE-A) conjugate (right peak) or staining with anti-IgG1 isotype control conjugated to phycoerythrin (PE-A-).

### Example 4: In Vitro Cytotoxicity Assays Using Human Cervical Carcinoma Cell Lines Co-Cultured with ICAM-1 CAR-T Cells

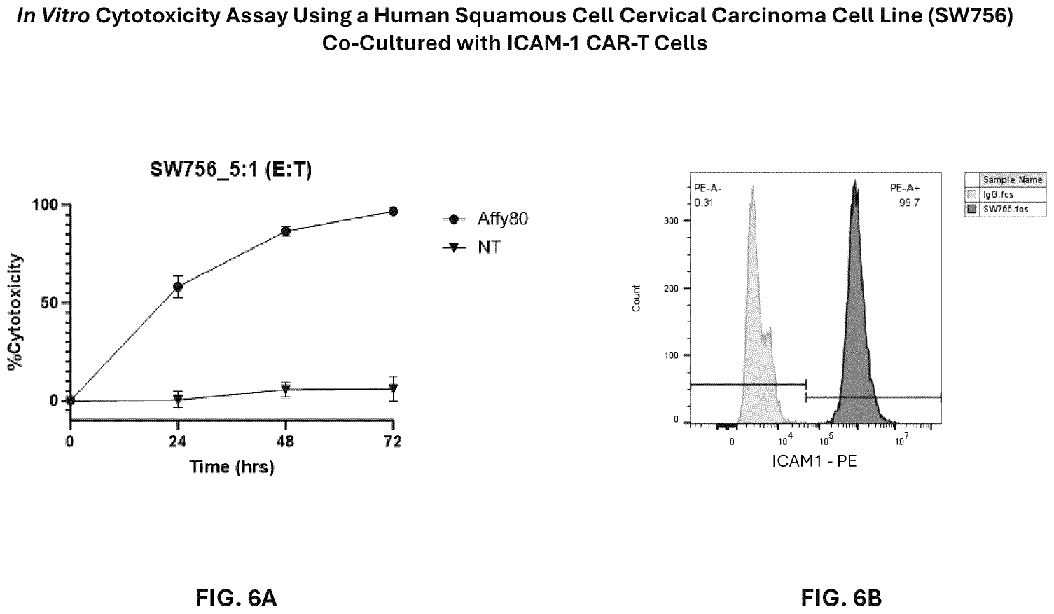

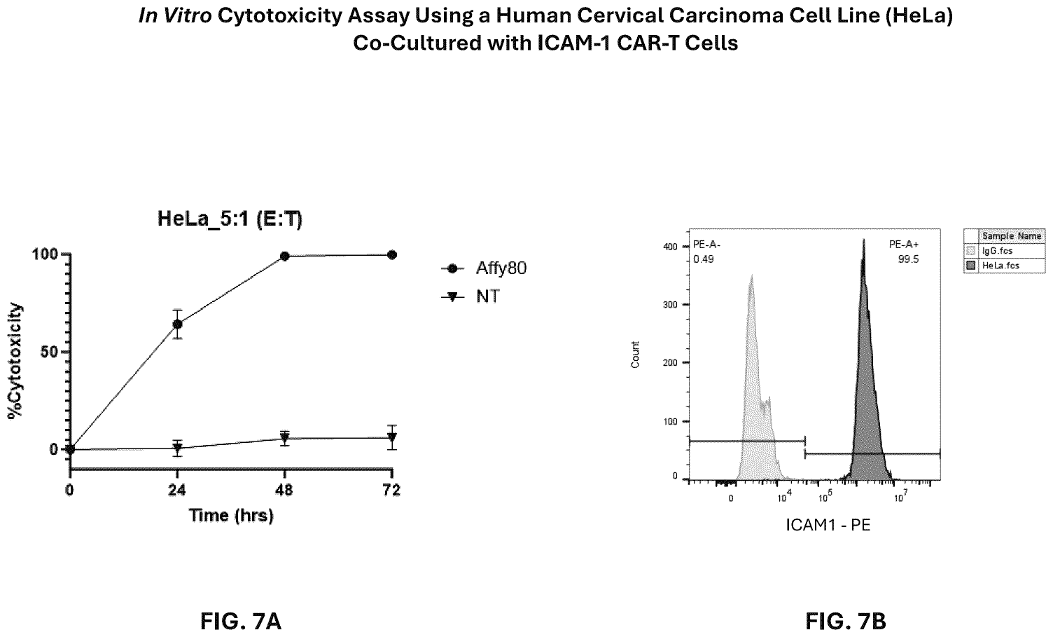

FIGS. 6-7 show the results from in vitro cytotoxicity assays using human cervical carcinoma cell lines co-cultured with ICAM-1 CAR-T cells at an effector:target (E:T) ratio of 5:1. The ICAM-1 CAR-T cells were produced by transfection with Affy80, a recombinant lentivirus expressing an ICAM-1 CAR as described in Example 1. The control cells were untransfected T cells (NT).

FIG. 6A is a graph showing % cytotoxicity as a function of time using a human squamous cell cervical carcinoma cell line (SW756) co-cultured with ICAM-1 CAR-T cells. FIG. 6B is a histogram showing expression of ICAM-1 in SW756 cells by flow cytometry following surface staining with an anti-ICAM-1 phycoerythrin (PE-A) conjugate (right peak) o or staining with anti-IgG1 isotype control conjugated to phycoerythrin (PE-A-). no staining (PE-A-). FIG. 7A is a graph showing % cytotoxicity as a function of time using a human cervical carcinoma cell line (HeLa) co-cultured with ICAM-1 CAR-T cells. FIG. 7B is a histogram showing expression of ICAM-1 in HeLa cells by flow cytometry following surface staining with an anti-ICAM-1 phycoerythrin (PE-A) conjugate (right peak) or staining with anti-IgG1 isotype control conjugated to phycoerythrin (PE-A-).

### Example 5: In Vitro Cytotoxicity Assays Using Human Hepatocellular Carcinoma Cell Lines Co-Cultured with ICAM-1 CAR-T Cells

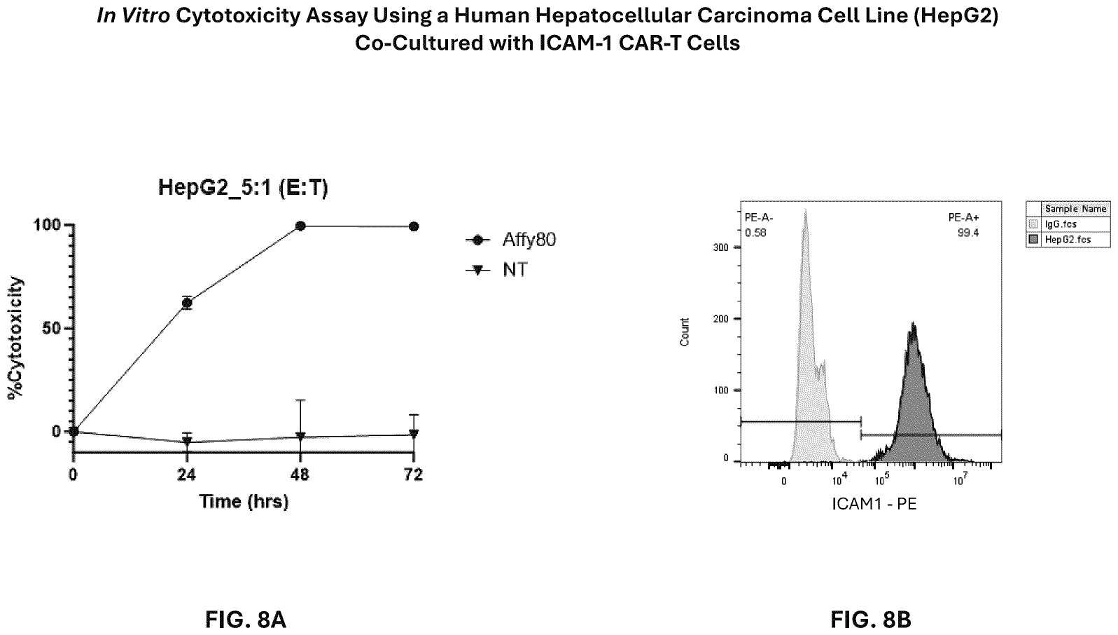

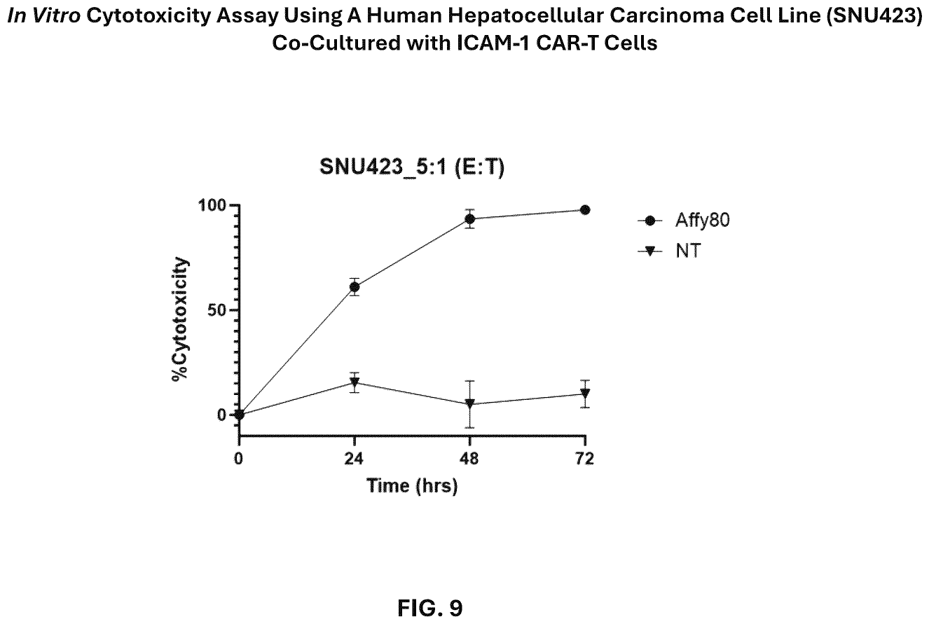

FIGS. 8-9 show the results from in vitro cytotoxicity assays using human hepatocellular carcinoma cell lines co-cultured with ICAM-1 CAR-T cells at an effector:target (E:T) ratio of 5:1. The ICAM-1 CAR-T cells were produced by transfection with Affy80, a recombinant lentivirus expressing an ICAM-1 CAR as described in Example 1. The control cells were untransfected T cells (NT).

FIG. 8A is a graph showing % cytotoxicity as a function of time using a human hepatocellular carcinoma cell lines (HepG2) co-cultured with ICAM-1 CAR-T cells. FIG. 8B is a histogram showing expression of ICAM-1 in HepG2 cells by flow cytometry following surface staining with an anti-ICAM-1 phycoerythrin (PE-A) conjugate (right peak) or staining with anti-IgG1 isotype control conjugated to phycoerythrin (PE-A-). FIG. 9 is a graph showing % cytotoxicity as a function of time using a human hepatocellular carcinoma cell line (SNU423) co-cultured with ICAM-1 CAR-T cells.

### Example 6: In Vitro Cytotoxicity Assays Using Human Squamous Cell Pharyngeal Carcinoma Cell Line Co-Cultured with ICAM-1 CAR-T Cells

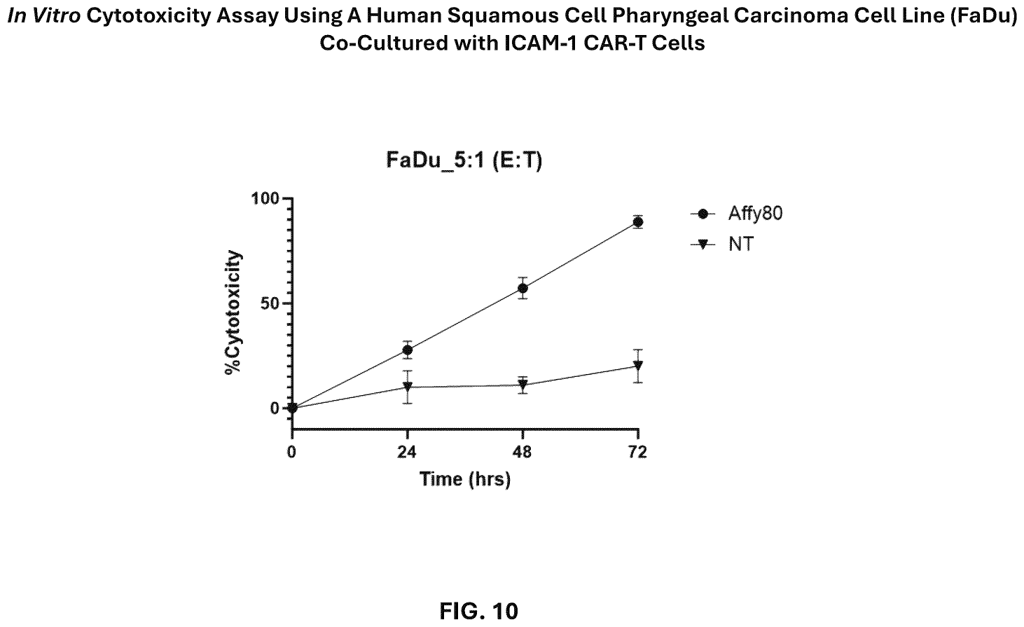

FIG. 10 is a graph showing % cytotoxicity as a function of time using a human squamous cell pharyngeal carcinoma cell line (FaDu) co-cultured with ICAM-1 CAR-T cells.

The results of the in vitro studies in Examples 3-6 show the efficacy of the ICAM-1 CAR-T cells against a variety of human carcinoma cells lines.

### Example 7: In Vivo Tumor Cell Killing of Human Lung Carcinoma Xenografts by ICAM-1 CAR-T Cells

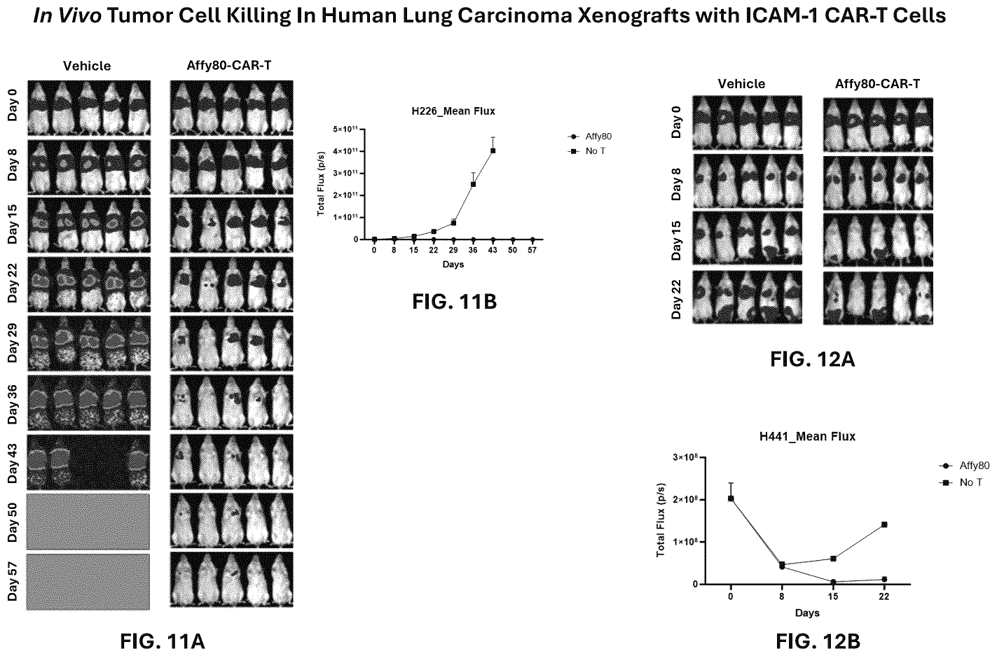

FIG. 11A shows images of H226 cell xenografts in mice after treatment with ICAM-1 CAR-T cells (right side, “Affy80-CAR-T”) or PBS (left side, “vehicle”) over a treatment period of 57 days. FIG. 11B is a graph showing greatly reduced imaging signals (total flux in photons/sec (p/s)) in H226 xenografts following treatment with ICAM-1 CAR-T cells (“Affy80”) as compared to vehicle only (PBS). Whereas all of the mice treated with the ICAM-1 CAR-T cells survived the tumor challenge and exhibited virtually complete elimination of tumors, tumors in the vehicle-treated control mice exhibited significant growth and all of the mice needed to be euthanized due to weight loss of over 20% (IACUC protocol).

FIG. 12A shows images of H441 cell xenografts in mice after treatment with ICAM-1 CAR-T cells (right side, “Affy80-CAR-T”) or PBS (left side, “vehicle”) over a treatment period of 22 days. FIG. 12B is a graph showing greatly reduced imaging signals in H441 xenografts following treatment with ICAM-1 CAR-T cells (“Affy80”) as compared to vehicle only (“No T”). As shown in FIG. 12A, mice treated with the ICAM-1 CAR-T cells showed greatly reduced imaging signals in all mice and non-detectable imaging signals in 2/5 mice. In contrast, the vehicle-treated mice exhibited progressively increased tumor growth as evidenced by increased imaging signals over the period of 22 days.

### Example 8: In Vivo Tumor Cell Killing of Human Cervical Carcinoma Xenografts by ICAM-1 CAR-T Cells

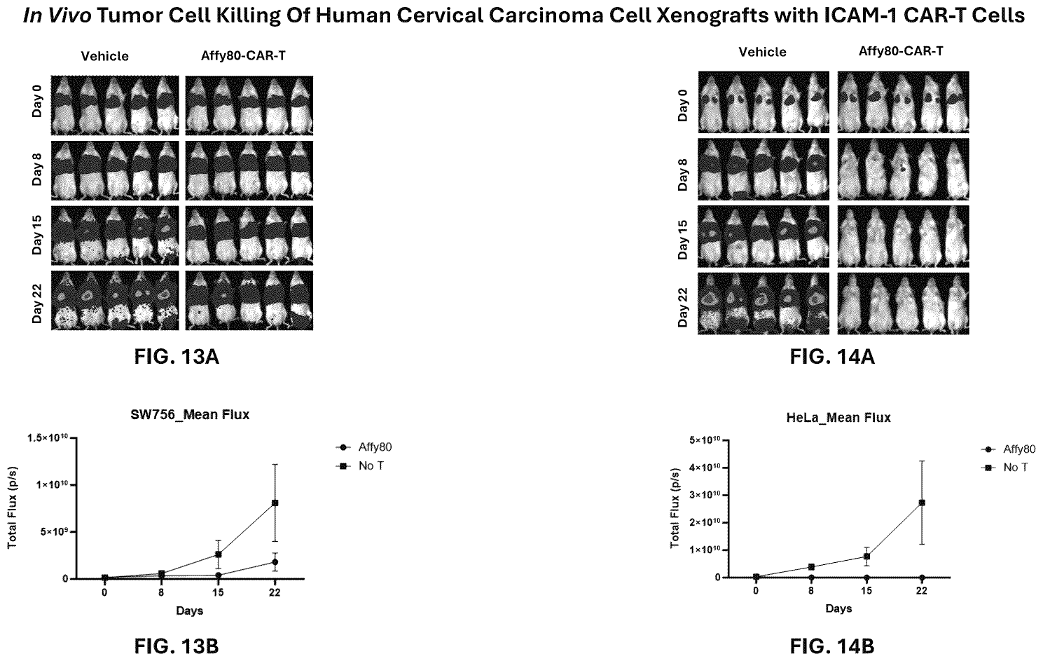

FIG. 13A shows images of SW756 cell xenografts in mice after treatment with ICAM-1 CAR-T cells (right side, “Affy80-CAR-T”) or PBS (left side, “vehicle”) over a treatment period of 22 days. FIG. 13B is a graph further reflecting the greatly reduced imaging signals in SW756 xenografts following treatment with ICAM-1 CAR-T cells (“Affy80”) as compared to the control mice (“No T”). The vehicle treated mice exhibited progressively increased tumor growth over the 22 day treatment period as evidenced by significantly increased imaging signals.

FIG. 14A shows images of HeLa cell xenografts in mice after treatment with ICAM-1 CAR-T cells (right side, “Affy80-CAR-T”) or PBS (left side, “vehicle”). FIG. 14B is a graph showing reduced imaging signals (total flux in photons/sec (p/s)) in H441 xenografts following treatment with ICAM-1 CAR-T cells (“Affy80”) as compared to the control mice (“No T”) over a treatment period of 22 days. As shown in FIG. 14A, mice treated with the ICAM-1 CAR-T cells showed virtually complete elimination of tumor cells by day 8. In contrast, the vehicle treated mice exhibited progressively increased tumor growth over the 22 day treatment period as evidenced by significantly increased imaging signals.

### Example 9: In Vitro Cytotoxicity Assays Using Primary Human Lung Epithelial Cells Co-Cultured with ICAM-1 CAR-T Cells

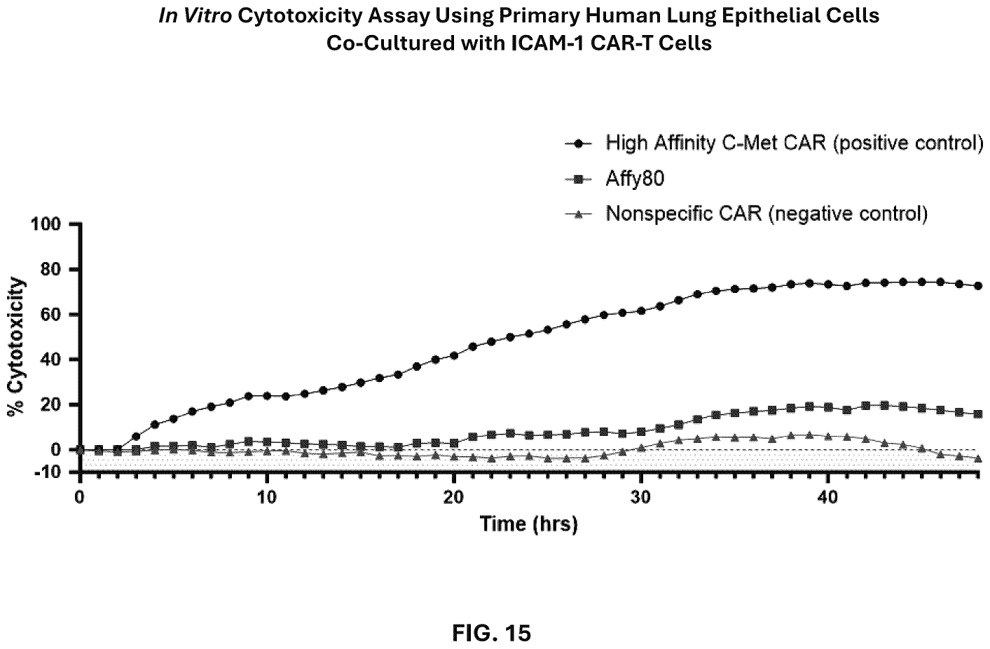

FIG. 15 is a graph showing % cytotoxicity as a function of time using primary human lung epithelial cells co-cultured at an effector:target (E:T) ratio of 5:1 with high affinity c-Met CAR-T cells, (positive control), low affinity ICAM-1 CAR-T cells (Affy80), or non-specific CD19 CAR-T cells. The % cytotoxicity results were normalized based on 39% CAR+ cells per T cell population compared to non-transduced lung epithelial cells. The results demonstrating significantly reduced toxicity of the low affinity ICAM-1 CAR-T cells against normal lung epithelial cells, but significantly high toxicity against ICAM-1 overexpressing lung epithelial (carcinoma) tumor cells as shown in the previous examples.

### Example 10: ICAM-1 Targeted CAR Constructs and Methods of Treatment

As described above, ICAM-1 is a cell surface glycoprotein that is overexpressed in, e.g., many solid tumors. AIC100 is a 3rd-generation CAR T cell with micromolar affinity to ICAM-1, and the binding affinity is tuned lower than most CARs used to date in preclinical (3) and clinical studies. Affinity tuned AIC100 CAR T cells are expected to selectively bind and kill tumor cells expressing cell surface ICAM-1 while sparing healthy cells. AIC100 is currently in the clinic (Phase 1) for anaplastic and poorly differentiated thyroid cancer (ATC/PDTC); NCT04420754. Advanced stage NSCLC has a high unmet medical need with patients having poor outcomes; 5-year survival of ˜10-20% (1,2). AIC100 may offer targeted treatment benefit to ICAM-1 positive NSCLC patients.

NSCLC tumor microarrays for adenocarcinoma (n=57) and squamous cell carcinoma (n=64) were stained for ICAM-1 and scored for membrane expression. Percentages of ICAM-1 expressing tumors were defined by H score with values >0 considered ICAM-1 positive and used to determine patient populations.

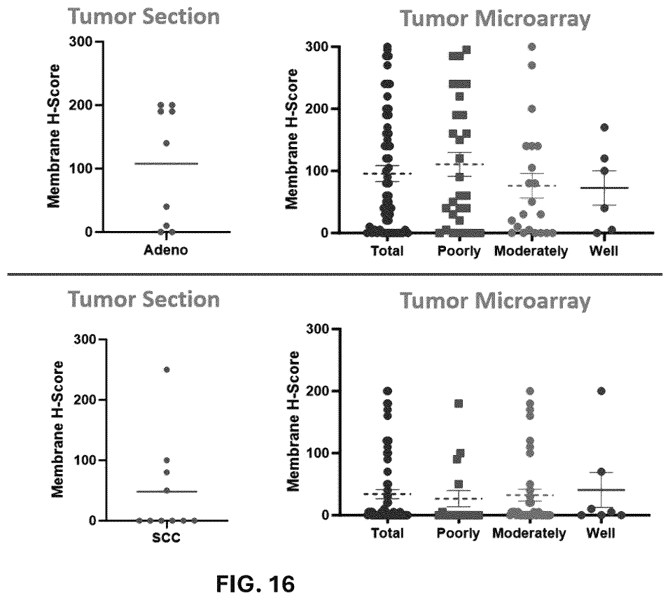

NSCLC has a high level of ICAM-1 membrane expression with approximately 77% of adenocarcinoma and 45% of squamous cell carcinoma patients having ICAM-1 expression. See FIG. 16 and Table 1.

TABLE 1

| | Adenocarcinoma | | Squamous Cell Carcinoma | |
|---|---|---|---|---|
| Category | Ratio | Percentage (%) | Ratio | Percentage (%) |
| Total Positive | 44/57 | 77.2 | 29/64 | 45.3 |
| Poor | 23/29 | 79.3 | 5/16 | 31.3 |
| Moderate | 15/21 | 71.4 | 18/38 | 47.4 |
| Well | 5/6 | 83.3 | 4/7 | 57.1 |
| Undeclared | 1/1 | 100.0 | 2/3 | 66.7 |

Preclinical efficacy using wild-type and ICAM-1 knock-out H226 cells were utilized in an in vitro cytotoxicity assay. Briefly, 5×104 target cells (H226) that are stably transduced to express GFP and firefly luciferase were co-cultured with CAR T cells at an effector to target ratio of 5:1. Co-cultures were carried out in T cell culture medium containing 150 ug/mL D-Luciferin and luminescence was measured every 24 hours for 3 days. To test preclinical safety primary human lung epithelial cells were co-cultured with AIC100, a high affinity c-Met CAR and a non-specific CD19 CAR at an effector to target ratio of 5:1.

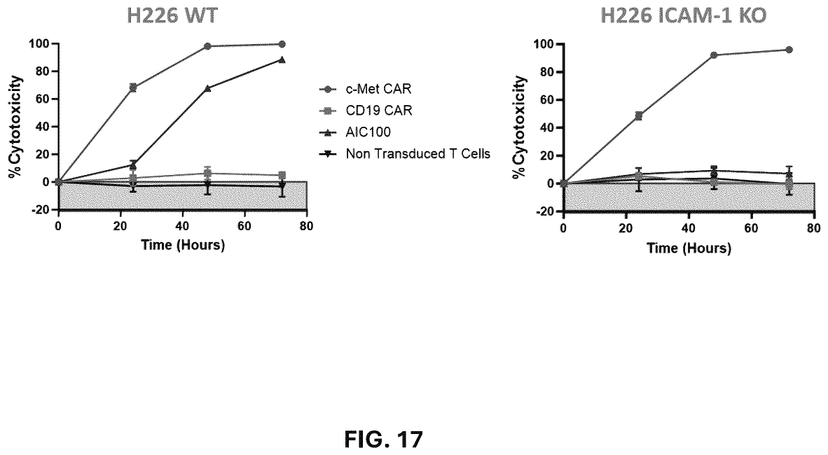

A high degree of cytotoxicity (97.1% at 72 hours post-exposure) was observed with AIC100 CAR T representative material in ICAM-1-expressing H226 cells (97.1%), similarly c-Met CAR T cells had a maximum cytotoxicity of 99.6%, whereas little to no activity was observed with CD19 CAR T cells and non-transduced cells (6.6% and −4.9%, respectively). In ICAM-1 knock-out H226 cells, no cytotoxicity was observed after 72 hours for CD19 and AIC100 CAR T cells whereas the c-Met CAR performed similarly to the WT. See FIG. 17.

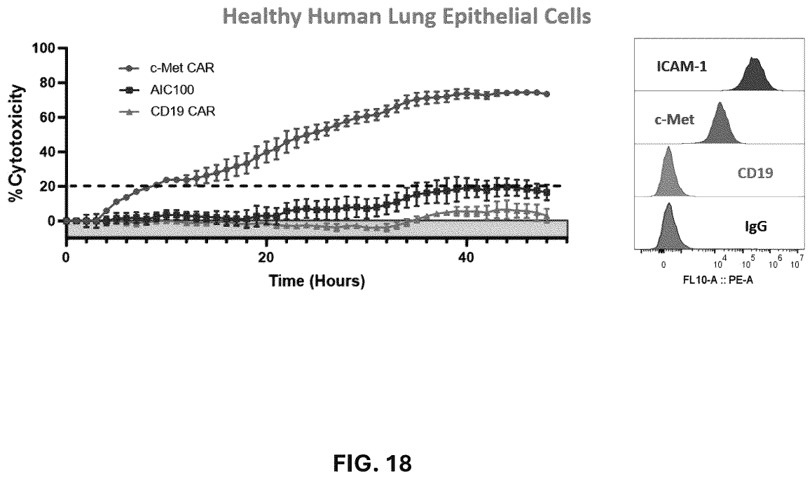

AIC100 and CD19 CAR T cells (negative control) had similar, limited cytotoxicity whereas high-affinity c-Met CAR T cells used as positive control had a high level of cytotoxicity in primary human lung epithelial cells. Normal lung epithelial cells which expressed ICAM-1 have limited engagement by AIC100 and are spared the cytotoxic activity of AIC100. See FIG. 18.

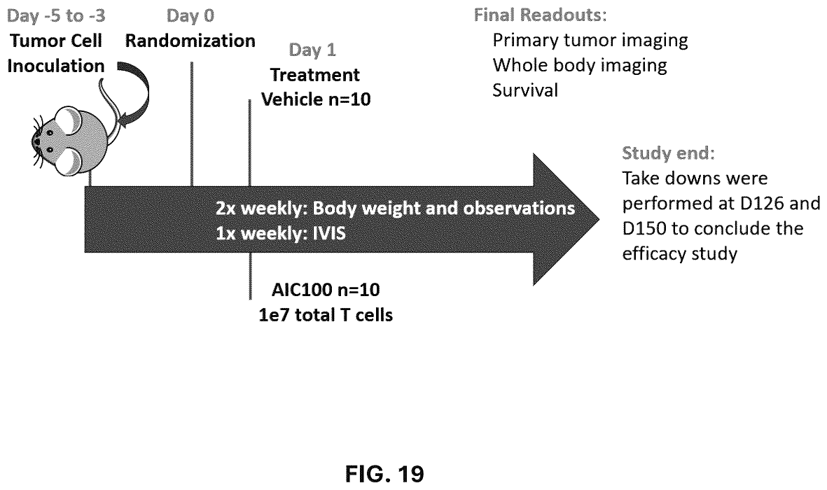

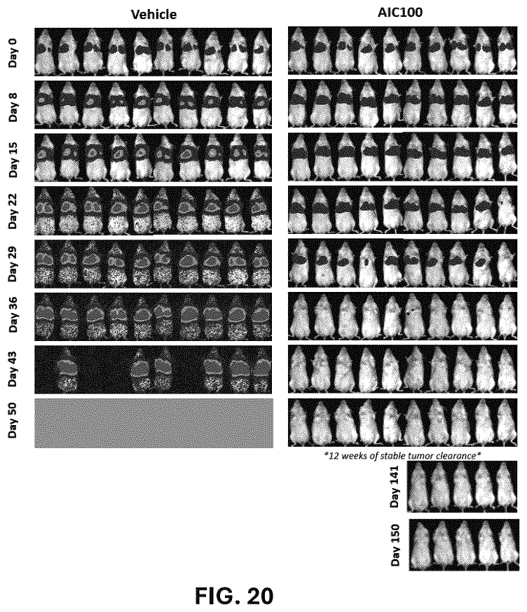

FIGS. 19 and 20 describe the in vivo study design. In vivo studies took place at Charles River Laboratories.

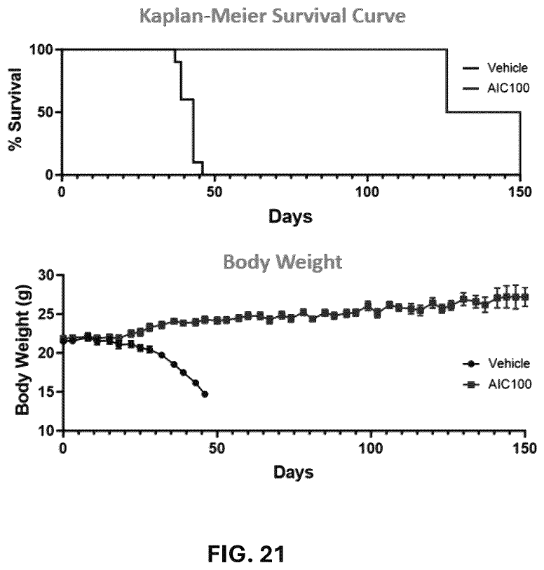

AIC100 CAR T cells eliminate tumor within 43 days post treatment and prevent relapse for at least 150 days. Animals treated with vehicle alone succumbed to disease by 46 days, whereas in AIC100 treated animals, tumors were eliminated and survival benefit of at least 150 days was observed with no tumor regression. AIC100 treated animals-maintained body weight throughout the duration of the study signifying a lack of toxicity. See FIG. 21.

TMA results show a significant proportion of NSCLC population are ICAM-1 positive. AIC100 successfully targeted and killed ICAM-1 positive tumor cells while having no toxic effect of ICAM-1 positive healthy primary human lung epithelial cells. AIC100 CAR T cells eliminated tumor within 43 days post treatment prevented relapse through study end (150 days). Animals treated with vehicle alone succumbed to disease within 46 days, whereas AIC100 treated animals survived until study end. AIC100 successfully eliminated NSCLC tumors and showed survival benefit in a xenograft disease model of NSCLC suggesting, there is clear potential for significant treatment benefit in the ICAM-1 positive population of NSCLC patients.

### REFERENCES

1. Li et al Front Immunol 2023; 14: 1195476. PMID 37559727
2. Thai et al Lancet 2021; 398: 535. PMID 34273294
3. Park et al Sci Rep 2017; 7(1): 14366. PMID 29085043

### LIST OF SEQUENCES

Exemplary amino acid sequences described herein are provided in Table 2 below:

TABLE 2
Amino Acid and Nucleotide Sequences for Exemplary Polypeptides Described
Herein

| SEQ | | |
|---|---|---|
| ID | | |
| NO: | Name | Sequence |
| 1 | Leukocyte function- | YNLDVRGARSFSPPRAGRHFGYRVLQVGNGVIVGAP |
| | associated molecule-1 | GEGNSTGSLYQCQSGTGHCLPVTLRGSNYTSKYLGM |
| | alpha subunit | TLATDPTDGSILACDPGLSRTCDQNTYLSGLCYLFRQN |
| | (LFA-1) alpha | LQGPMLQGRPGFQECIKGNVDLVFLFDGSMSLQPDEF |
| | (αL subunit) | QKILDFMKDVMKKLSNTSYQFAAVQFSTSYKTEFDFS |
| | | DYVKWKDPDALLKHVKHMLLLTNTFGAINYVATEVF |
| | | REELGARPDATKVLIIITDGEATDSGNIDAAKDIIRYIIG |
| | | IGKHFQTKESQETLHKFASKPASEFVKILDTFEKLKDL |
| | | FTELQKKIYVIEGTSKQDLTSFNMELSSSGISADLSRGH |
| | | AVVGAVGAKDWAGGFLDLKADLQDDTFIGNEPLTPE |
| | | VRAGYLGYTVTWLPSRQKTSLLASGAPRYQHMGRVL |
| | | LFQEPQGGGHWSQVQTIHGTQIGSYFGGELCGVDVD |
| | | QDGETELLLIGAPLFYGEQRGGRVFIYQRRQLGFEEVS |
| | | ELQGDPGYPLGRFGEAITALTDINGDGLVDVAVGAPL |
| | | EEQGAVYIFNGRHGGLSPQPSQRIEGTQVLSGIQWFGR |
| | | SIHGVKDLEGDGLADVAVGAESQMIVLSSRPVVDMV |
| | | TLMSFSPAEIPVHEVECSYSTSNKMKEGVNITICFQIKS |
| | | LYPQFQGRLVANLTYTLQLDGHRTRRRGLFPGGRHEL |
| | | RRNIAVTTSMSCTDFSFHFPVCVQDLISPINVSLNFSLW |
| | | EEEGTPRDQRAQGKDIPPILRPSLHSETWEIPFEKNCGE |
| | | DKKCEANLRVSFSPARSRALRLTAFASLSVELSLSNLE |
| | | EDAYWVQLDLHFPPGLSFRKVEMLKPHSQIPVSCEEL |
| | | PEESRLLSRALSCNVSSPIFKAGHSVALQMMENTLVNS |
| | | SWGDSVELHANVTCNNEDSDLLEDNSATTIIPILYPINI |
| | | LIQDQEDSTLYVSFTPKGPKIHQVKHMYQVRIQPSIHD |
| | | HNIPTLEAVVGVPQPPSEGPITHQWSVQMEPPVPCHYE |
| | | DLERLPDAAEPCLPGALFRCPVVFRQEILVQVIGTLEL |
| | | VGEIEASSMFSLCSSLSISFNSSKHFHLYGSNASLAQVV |
| | | MKVDVVYEKQMLYLYVLSGIGGLLLLLLIFIVLYKVG |
| | | FFKRNLKEKMEAGRGVPNGIPAEDSEQLASGQEAGDP |
| | | GCLKPLHEKDSESGGGKD |
| 2 | I domain of αL subunit | GNVDLVFLFDGSMSLQPDEFQKILDFMKDVMKKLSN |
| | of LFA-1 (wt) | TSYQFAAVQFSTSYKTEFDFSDYVKWKDPDALLKHV |
| | (aa 128-311) | KHMLLLTNTFGAINYVATEVFREELGARPDATKVLIII |
| | | TDGEATDSGNIDAAKDIIRYIIGIGKHFQTKESQETLHK |
| | | FASKPASEFVKILDTFEKLKDLFTELQKKIYVIEG |
| 3 | Mutant I domain of αL | GNVDLVFLFDGSMSLQPDEFQKILDFMKDVMKKLSN |
| | subunit of LFA-1 | TSYQFAAVQFSTSYKTEFDFSDYVKWKDPDALLKHV |
| | (F292A) | KHMLLLTNTFGAINYVATEVFREELGARPDATKVLIII |
| | | TDGEATDSGNIDAAKDIIRYIIGIGKHFQTKESQETLHK |
| | | FASKPASEFVKILDTAEKLKDLFTELQKKIYVIEG |
| 4 | CD8 alpha | IYIWAPLAGTCGVLLLSLVITLYC |
| | transmembrane domain | |
| 5 | CD28 transmembrane | FWVLVVVGGVLACYSLLVTVAFII |
| | domain | |
| 6 | ICOS transmembrane | FWLPIGCAAFVVVCILGCILICWL |
| | domain | |
| 7 | GITR transmembrane | LGWLTVVLLAVAACVLLLTSAQLGL |
| | domain | |
| 8 | 4-1BB costimulatory | KRGRKKLLYIFKQPFMRPVQTTQEEDGCSCRFPEEEE |
| | domain | GGCEL |
| 9 | CD28 costimulatory | RSKRSRLLHSDYMNMTPRRPGPTRKHYQPYAPPRDFA |
| | domain | AYRS |
| 10 | OX-40 costimulatory | ALYLLRRDQRLPPDAHKPPGGGSFRTPIQEEQADAHS |
| | domain | TLAKI |
| 11 | CD3 zeta signaling | RVKFSRSADAPAYKQGQNQLYNELNLGRREEYDVLD |
| | domain | KRRGRDPEMGGKPRRKNPQEGLYNELQKDKMAEAY |
| | | SEIGMKGERRRGKGHDGLYQGLSTATKDTYDALHMQ |
| | | ALPPR |
| 12 | CD8 alpha hinge | TTTPAPRPPTPAPTIASQPLSLRPEACRPAAGGAVHTR |
| | | GLDFACD |
| 13 | CD28 hinge | RAAAIEVMYPPPYLDNEKSNGTIIHVKGKHLCPSPLFP |
| | | GPSKPKDPK |
| 14 | IgG4 hinge | SKYGPPCPSCP |
| 15 | c-myc tag | GSEQKLISEEDL |
| 16 | SSTR2 (aa1-381) | MDMADEPLNGSHTWLSIPFDLNGSVVSTNTSNQTEPY |
| | | YDLTSNAVLTFIYFVVCIIGLCGNTLVIYVILRYAKMK |
| | | TITNIYILNLAIADELFMLGLPFLAMQVALVHWPFGKA |
| | | ICRVVMTVDGINQFTSIFCLTVMSIDRYLAVVHPIKSA |
| | | KWRRPRTAKMITMAVWGVSLLVILPIMIYAGLRSNQ |
| | | WGRSSCTINWPGESGAWYTGFIIYTFILGFLVPLTIICL |
| | | CYLFIIIKVKSSGIRVGSSKRKKSEKKVTRMVSIVVAVF |
| | | IFCWLPFYIFNVSSVSMAISPTPALKGMFDFVVVLTYA |
| | | NSCANPILYAFLSDNFKKSFQNVLCLVKVSGTDDGER |
| | | SDSKQDKSRLNETTETQRTLLNGDLQTSI |
| 17 | SSTR2 (aa 1-314) | MDMADEPLNGSHTWLSIPFDLNGSVVSTNTSNQTEPY |
| | | YDLTSNAVLTFIYFVVCIIGLCGNTLVIYVILRYAKMK |
| | | TITNIYILNLAIADELFMLGLPFLAMQVALVHWPFGKA |
| | | ICRVVMTVDGINQFTSIFCLTVMSIDRYLAVVHPIKSA |
| | | KWRRPRTAKMITMAVWGVSLLVILPIMIYAGLRSNQ |
| | | WGRSSCTINWPGESGAWYTGFIIYTFILGFLVPLTIICL |
| | | CYLFIIIKVKSSGIRVGSSKRKKSEKKVTRMVSIVVAVF |
| | | IFCWLPFYIFNVSSVSMAISPTPALKGMFDFVVVLTYA |
| | | NSCANPILYAF |
| 18 | CD8 alpha chain signal | MALPVTALLLPLALLLHAARP |
| | peptide | |
| 19 | (GGG)n linker | (GGGS)n |
| 20 | (GGGGS)x3 linker | GGGGSGGGGSGGGGS |
| 21 | P2A peptide | GSGATNFSLLKQAGDVEENPGP |
| 22 | E2A peptide | GSGQCTNYALLKLAGDVESNPGP |
| 23 | T2A peptide | GSGEGRGSLLTCGDVEENPGP |
| 24 | F2A peptide | GSGVKQTLNFDLLKLAGDVESNPGP |
| 25 | ICAM-1 CAR only | GGCAACGTAGACCTGGTATTTCTGTTTGATGGTTCG |
| | | ATGAGCTTGCAGCCAGATGAATTTCAGAAAATTCTG |
| | | GACTTCATGAAGGATGTGATGAAGAAACTCAGCAA |
| | | CACTTCGTACCAGTTTGCTGCTGTTCAGTTTTCCACA |
| | | AGCTACAAAACAGAATTTGATTTCTCAGATTATGTT |
| | | AAATGGAAGGACCCTGATGCTCTGCTGAAGCATGT |
| | | AAAGCACATGTTGCTGTTGACCAATACCTTTGGTGC |
| | | CATCAATTATGTCGCGACAGAGGTGTTCCGGGAGG |
| | | AGCTGGGGGCCCGGCCAGATGCCACCAAAGTGCTT |
| | | ATCATCATCACGGATGGGGAGGCCACTGACAGTGG |
| | | CAACATCGATGCGGCCAAAGACATCATCCGCTACA |
| | | TCATCGGGATTGGAAAGCATTTTCAGACCAAGGAG |
| | | AGTCAGGAGACCCTCCACAAATTTGCATCAAAACC |
| | | CGCGAGCGAGTTTGTGAAAATTCTGGACACAGCTG |
| | | AGAAGCTGAAAGATCTATTCACTGAGCTGCAGAAG |
| | | AAGATCTATGTCATTGAGGGCGCTAGCACGACCAC |
| | | TCCGGCGCCGCGCCCACCGACTCCGGCCCCAACTAT |
| | | CGCGAGCCAGCCCCTGTCGCTGAGGCCGGAAGCAT |
| | | GCCGCCCTGCCGCCGGAGGTGCTGTGCATACCCGG |
| | | GGATTGGACTTCGCATGCGACTTTTGGGTGCTGGTG |
| | | GTGGTTGGTGGAGTCCTGGCTTGCTATAGCTTGCTA |
| | | GTAACAGTGGCCTTTATTATTTTCTGGGTGAGGAGT |
| | | AAGAGGAGCAGGCTCCTGCACAGTGACTACATGAA |
| | | CATGACTCCCCGCCGCCCCGGGCCCACCCGCAAGC |
| | | ATTACCAGCCCTATGCCCCACCACGCGACTTCGCAG |
| | | CCTATCGCTCCAAGCGGGGTCGGAAAAAGCTTCTGT |
| | | ACATTTTCAAGCAGCCCTTCATGAGGCCCGTGCAAA |
| | | CCACCCAGGAGGAGGACGGTTGCTCCTGCCGGTTC |
| | | CCCGAAGAGGAAGAAGGAGGTTGCGAGCTGAGAGT |
| | | GAAGTTCAGCAGGAGCGCAGACGCCCCCGCGTACC |
| | | AGCAGGGCCAGAACCAGCTCTATAACGAGCTCAAT |
| | | CTAGGACGAAGAGAGGAGTACGATGTTTTGGACAA |
| | | GAGACGTGGCCGGGACCCTGAGATGGGGGGAAAGC |
| | | CGAGAAGGAAGAACCCTCAGGAAGGCCTGTACAAT |
| | | GAACTGCAGAAAGATAAGATGGCGGAGGCCTACAG |
| | | TGAGATTGGGATGAAAGGCGAGCGCCGGAGGGGCA |
| | | AGGGGCACGATGGCCTTTACCAGGGTCTCAGTACA |
| | | GCCACCAAGGACACCTACGACGCCCTTCACATGCA |
| | | GGCCCTGCCCCCTCGC |
| 26 | Affy80 | ATGGCCTTACCAGTGACCGCCTTGCTCCTGCCGCTG |
| | | GCCTTGCTGCTCCACGCCGCCAGGCCGGGATCCGA |
| | | ACAAAAACTCATCTCAGAAGAGGATCTGGGATCGG |
| | | GCAACGTAGACCTGGTATTTCTGTTTGATGGTTCGA |
| | | TGAGCTTGCAGCCAGATGAATTTCAGAAAATTCTGG |
| | | ACTTCATGAAGGATGTGATGAAGAAACTCAGCAAC |
| | | ACTTCGTACCAGTTTGCTGCTGTTCAGTTTTCCACA |
| | | AGCTACAAAACAGAATTTGATTTCTCAGATTATGTT |
| | | AAATGGAAGGACCCTGATGCTCTGCTGAAGCATGT |
| | | AAAGCACATGTTGCTGTTGACCAATACCTTTGGTGC |
| | | CATCAATTATGTCGCGACAGAGGTGTTCCGGGAGG |
| | | AGCTGGGGGCCCGGCCAGATGCCACCAAAGTGCTT |
| | | ATCATCATCACGGATGGGGAGGCCACTGACAGTGG |
| | | CAACATCGATGCGGCCAAAGACATCATCCGCTACA |
| | | TCATCGGGATTGGAAAGCATTTTCAGACCAAGGAG |
| | | AGTCAGGAGACCCTCCACAAATTTGCATCAAAACC |
| | | CGCGAGCGAGTTTGTGAAAATTCTGGACACAGCTG |
| | | AGAAGCTGAAAGATCTATTCACTGAGCTGCAGAAG |
| | | AAGATCTATGTCATTGAGGGCGCTAGCACGACCAC |
| | | TCCGGCGCCGCGCCCACCGACTCCGGCCCCAACTAT |
| | | CGCGAGCCAGCCCCTGTCGCTGAGGCCGGAAGCAT |
| | | GCCGCCCTGCCGCCGGAGGTGCTGTGCATACCCGG |
| | | GGATTGGACTTCGCATGCGACTTTTGGGTGCTGGTG |
| | | GTGGTTGGTGGAGTCCTGGCTTGCTATAGCTTGCTA |
| | | GTAACAGTGGCCTTTATTATTTTCTGGGTGAGGAGT |
| | | AAGAGGAGCAGGCTCCTGCACAGTGACTACATGAA |
| | | CATGACTCCCCGCCGCCCCGGGCCCACCCGCAAGC |
| | | ATTACCAGCCCTATGCCCCACCACGCGACTTCGCAG |
| | | CCTATCGCTCCAAGCGGGGTCGGAAAAAGCTTCTGT |
| | | ACATTTTCAAGCAGCCCTTCATGAGGCCCGTGCAAA |
| | | CCACCCAGGAGGAGGACGGTTGCTCCTGCCGGTTC |
| | | CCCGAAGAGGAAGAAGGAGGTTGCGAGCTGAGAGT |
| | | GAAGTTCAGCAGGAGCGCAGACGCCCCCGCGTACC |
| | | AGCAGGGCCAGAACCAGCTCTATAACGAGCTCAAT |
| | | CTAGGACGAAGAGAGGAGTACGATGTTTTGGACAA |
| | | GAGACGTGGCCGGGACCCTGAGATGGGGGGAAAGC |
| | | CGAGAAGGAAGAACCCTCAGGAAGGCCTGTACAAT |
| | | GAACTGCAGAAAGATAAGATGGCGGAGGCCTACAG |
| | | TGAGATTGGGATGAAAGGCGAGCGCCGGAGGGGCA |
| | | AGGGGCACGATGGCCTTTACCAGGGTCTCAGTACA |
| | | GCCACCAAGGACACCTACGACGCCCTTCACATGCA |
| | | GGCCCTGCCCCCTCGCACTAGTGGAAGCGGAGCTA |
| | | CTAACTTCAGCCTGCTGAAGCAGGCTGGAGACGTG |
| | | GAGGAGAACCCTGGACCTTCTAGAATGGACATGGC |
| | | GGATGAGCCACTCAATGGAAGCCACACATGGCTAT |
| | | CCATTCCATTTGACCTCAATGGCTCTGTGGTGTCAA |
| | | CCAACACCTCAAACCAGACAGAGCCGTACTATGAC |
| | | CTGACAAGCAATGCAGTCCTCACATTCATCTATTTT |
| | | GTGGTCTGCATCATTGGGTTGTGTGGCAACACACTT |
| | | GTCATTTATGTCATCCTCCGCTATGCCAAGATGAAG |
| | | ACCATCACCAACATTTACATCCTCAACCTGGCCATC |
| | | GCAGATGAGCTCTTCATGCTGGGTCTGCCTTTCTTG |
| | | GCTATGCAGGTGGCTCTGGTCCACTGGCCCTTTGGC |
| | | AAGGCCATTTGCCGGGTGGTCATGACTGTGGATGG |
| | | CATCAATCAGTTCACCAGCATCTTCTGCCTGACAGT |
| | | CATGAGCATCGACCGATACCTGGCTGTGGTCCACCC |
| | | CATCAAGTCGGCCAAGTGGAGGAGACCCCGGACGG |
| | | CCAAGATGATCACCATGGCTGTGTGGGGAGTCTCTC |
| | | TGCTGGTCATCTTGCCCATCATGATATATGCTGGGC |
| | | TCCGGAGCAACCAGTGGGGGAGAAGCAGCTGCACC |
| | | ATCAACTGGCCAGGTGAATCTGGGGCTTGGTACAC |
| | | AGGGTTCATCATCTACACTTTCATTCTGGGGTTCCT |
| | | GGTACCCCTCACCATCATCTGTCTTTGCTACCTGTTC |
| | | ATTATCATCAAGGTGAAGTCCTCTGGAATCCGAGTG |
| | | GGCTCCTCTAAGAGGAAGAAGTCTGAGAAGAAGGT |
| | | CACCCGAATGGTGTCCATCGTGGTGGCTGTCTTCAT |
| | | CTTCTGCTGGCTTCCCTTCTACATATTCAACGTTTCT |
| | | TCCGTCTCCATGGCCATCAGCCCCACCCCAGCCCTT |
| | | AAAGGCATGTTTGACTTTGTGGTGGTCCTCACCTAT |
| | | GCTAACAGCTGTGCCAACCCTATCCTATATGCCTTC |
| | | TTGTCTGACAACTTCAAGAAGAGCTTCCAGAATGTC |
| | | CTCTGCTTGGTCAAGGTGAGCGGCACAGATGATGG |
| | | GGAGCGGAGTGACAGTAAGCAGGACAAATCCCGGC |
| | | TGAATGAGACCACGGAGACCCAGAGGACCCTCCTC |
| | | AATGGAGACCTCCAAACCAGTATCTAA |
| 27 | ICAM-1 CAR only | GNVDLVFLFDGSMSLQPDEFQKILDFMKDVMKKLSN |
| | (no signal, myc-tag or | TSYQFAAVQFSTSYKTEFDFSDYVKWKDPDALLKHV |
| | SSTR2) | KHMLLLTNTFGAINYVATEVFREELGARPDATKVLIII |
| | | TDGEATDSGNIDAAKDIIRYIIGIGKHFQTKESQETLHK |
| | | FASKPASEFVKILDTAEKLKDLFTELQKKIYVIEGASTT |
| | | TPAPRPPTPAPTIASQPLSLRPEACRPAAGGAVHTRGL |
| | | DFACDFWVLVVVGGVLACYSLLVTVAFIIFWVRSKRS |
| | | RLLHSDYMNMTPRRPGPTRKHYQPYAPPRDFAAYRS |
| | | KRGRKKLLYIFKQPFMRPVQTTQEEDGCSCRFPEEEE |
| | | GGCELRVKFSRSADAPAYQQGQNQLYNELNLGRREE |
| | | YDVLDKRRGRDPEMGGKPRRKNPQEGLYNELQKDK |
| | | MAEAYSEIGMKGERRRGKGHDGLYQGLSTATKDTYD |
| | | ALHMQALPPR |
| 28 | ICAM-1 CAR-SSTR2 | GNVDLVFLFDGSMSLQPDEFQKILDFMKDVMKKLSN |
| | fusion protein | TSYQFAAVQFSTSYKTEFDFSDYVKWKDPDALLKHV |
| | (no signal or myc-tag) | KHMLLLTNTFGAINYVATEVFREELGARPDATKVLIII |
| | | TDGEATDSGNIDAAKDIIRYIIGIGKHFQTKESQETLHK |
| | | FASKPASEFVKILDTAEKLKDLFTELQKKIYVIEGASTT |
| | | TPAPRPPTPAPTIASQPLSLRPEACRPAAGGAVHTRGL |
| | | DFACDFWVLVVVGGVLACYSLLVTVAFIIFWVRSKRS |
| | | RLLHSDYMNMTPRRPGPTRKHYQPYAPPRDFAAYRS |
| | | KRGRKKLLYIFKQPFMRPVQTTQEEDGCSCRFPEEEE |
| | | GGCELRVKFSRSADAPAYQQGQNQLYNELNLGRREE |
| | | YDVLDKRRGRDPEMGGKPRRKNPQEGLYNELQKDK |
| | | MAEAYSEIGMKGERRRGKGHDGLYQGLSTATKDTYD |
| | | ALHMQALPPRTSGSGATNFSLLKQAGDVEENPGPSRM |
| | | DMADEPLNGSHTWLSIPFDLNGSVVSTNTSNQTEPYY |
| | | DLTSNAVLTFIYFVVCIIGLCGNTLVIYVILRYAKMKTI |
| | | TNIYILNLAIADELFMLGLPFLAMQVALVHWPFGKAIC |
| | | RVVMTVDGINQFTSIFCLTVMSIDRYLAVVHPIKSAK |
| | | WRRPRTAKMITMAVWGVSLLVILPIMIYAGLRSNQW |
| | | GRSSCTINWPGESGAWYTGFIIYTFILGFLVPLTIICLCY |
| | | LFIIIKVKSSGIRVGSSKRKKSEKKVTRMVSIVVAVFIF |
| | | CWLPFYIFNVSSVSMAISPTPALKGMFDFVVVLTYAN |
| | | SCANPILYAFLSDNFKKSFQNVLCLVKVSGTDDGERS |
| | | DSKQDKSRLNETTETQRTLLNGDLQTSI |
| 29 | ICAM-1 CAR-SSTR2 | MALPVTALLLPLALLLHAARPGSEQKLISEEDLGSGN |
| | fusion protein | VDLVFLFDGSMSLQPDEFQKILDFMKDVMKKLSNTSY |
| | (with signal peptide | QFAAVQFSTSYKTEFDFSDYVKWKDPDALLKHVKHM |
| | and c-myc tag) | LLLTNTFGAINYVATEVFREELGARPDATKVLIIITDGE |
| | | ATDSGNIDAAKDIIRYIIGIGKHFQTKESQETLHKFASK |
| | | PASEFVKILDTAEKLKDLFTELQKKIYVIEGASTTTPAP |
| | | RPPTPAPTIASQPLSLRPEACRPAAGGAVHTRGLDFAC |
| | | DFWVLVVVGGVLACYSLLVTVAFIIFWVRSKRSRLLH |
| | | SDYMNMTPRRPGPTRKHYQPYAPPRDFAAYRSKRGR |
| | | KKLLYIFKQPFMRPVQTTQEEDGCSCRFPEEEEGGCEL |
| | | RVKFSRSADAPAYQQGQNQLYNELNLGRREEYDVLD |
| | | KRRGRDPEMGGKPRRKNPQEGLYNELQKDKMAEAY |
| | | SEIGMKGERRRGKGHDGLYQGLSTATKDTYDALHMQ |
| | | ALPPRTSGSGATNFSLLKQAGDVEENPGPSRMDMADE |
| | | PLNGSHTWLSIPFDLNGSVVSTNTSNQTEPYYDLTSNA |
| | | VLTFIYFVVCIIGLCGNTLVIYVILRYAKMKTITNIYILN |
| | | LAIADELFMLGLPFLAMQVALVHWPFGKAICRVVMT |
| | | VDGINQFTSIFCLTVMSIDRYLAVVHPIKSAKWRRPRT |
| | | AKMITMAVWGVSLLVILPIMIYAGLRSNQWGRSSCTI |
| | | NWPGESGAWYTGFIIYTFILGFLVPLTIICLCYLFIIIKV |
| | | KSSGIRVGSSKRKKSEKKVTRMVSIVVAVFIFCWLPFY |
| | | IFNVSSVSMAISPTPALKGMFDFVVVLTYANSCANPIL |
| | | YAFLSDNFKKSFQNVLCLVKVSGTDDGERSDSKQDKS |
| | | RLNETTETQRTLLNGDLQTSI |
| 30 | ICAM-1 CAR-SSTR2 | GSEQKLISEEDLGSGNVDLVFLFDGSMSLQPDEFQKIL |
| | fusion protein | DFMKDVMKKLSNTSYQFAAVQFSTSYKTEFDFSDYV |
| | (minus signal peptide | KWKDPDALLKHVKHMLLLTNTFGAINYVATEVFREE |
| | with myc-tag | LGARPDATKVLIIITDGEATDSGNIDAAKDIIRYIIGIGK |
| | | HFQTKESQETLHKFASKPASEFVKILDTAEKLKDLFTE |
| | | LOKKIYVIEGASTTTPAPRPPTPAPTIASQPLSLRPEACR |
| | | PAAGGAVHTRGLDFACDFWVLVVVGGVLACYSLLV |
| | | TVAFIIFWVRSKRSRLLHSDYMNMTPRRPGPTRKHYQ |
| | | PYAPPRDFAAYRSKRGRKKLLYIFKQPFMRPVQTTQE |
| | | EDGCSCRFPEEEEGGCELRVKFSRSADAPAYQQGQNQ |
| | | LYNELNLGRREEYDVLDKRRGRDPEMGGKPRRKNPQ |
| | | EGLYNELQKDKMAEAYSEIGMKGERRRGKGHDGLY |
| | | QGLSTATKDTYDALHMQALPPRTSGSGATNFSLLKQA |
| | | GDVEENPGPSRMDMADEPLNGSHTWLSIPFDLNGSVV |
| | | STNTSNQTEPYYDLTSNAVLTFIYFVVCIIGLCGNTLVI |
| | | YVILRYAKMKTITNIYILNLAIADELFMLGLPFLAMQV |
| | | ALVHWPFGKAICRVVMTVDGINQFTSIFCLTVMSIDR |
| | | YLAVVHPIKSAKWRRPRTAKMITMAVWGVSLLVILPI |
| | | MIYAGLRSNQWGRSSCTINWPGESGAWYTGFIIYTFIL |
| | | GFLVPLTIICLCYLFIIIKVKSSGIRVGSSKRKKSEKKVT |
| | | RMVSIVVAVFIFCWLPFYIFNVSSVSMAISPTPALKGM |
| | | FDFVVVLTYANSCANPILYAFLSDNFKKSFQNVLCLV |
| | | KVSGTDDGERSDSKQDKSRLNETTETQRTLLNGDLQT |
| | | SI |
| 31 | ICAM-1 CAR-SSTR2 | GNVDLVFLFDGSMSLQPDEFQKILDFMKDVMKKLSN |
| | fusion protein | TSYQFAAVQFSTSYKTEFDFSDYVKWKDPDALLKHV |
| | (minus signal peptide | KHMLLLTNTFGAINYVATEVFREELGARPDATKVLIII |
| | and myc-tag | TDGEATDSGNIDAAKDIIRYIIGIGKHFQTKESQETLHK |
| | | FASKPASEFVKILDTAEKLKDLFTELQKKIYVIEGASTT |
| | | TPAPRPPTPAPTIASQPLSLRPEACRPAAGGAVHTRGL |
| | | DFACDFWVLVVVGGVLACYSLLVTVAFIIFWVRSKRS |
| | | RLLHSDYMNMTPRRPGPTRKHYQPYAPPRDFAAYRS |
| | | KRGRKKLLYIFKQPFMRPVQTTQEEDGCSCRFPEEEE |
| | | GGCELRVKFSRSADAPAYQQGQNQLYNELNLGRREE |
| | | YDVLDKRRGRDPEMGGKPRRKNPQEGLYNELQKDK |
| | | MAEAYSEIGMKGERRRGKGHDGLYQGLSTATKDTYD |
| | | ALHMQALPPRTSGSGATNFSLLKQAGDVEENPGPSRM |
| | | DMADEPLNGSHTWLSIPFDLNGSVVSTNTSNQTEPYY |
| | | DLTSNAVLTFIYFVVCIIGLCGNTLVIYVILRYAKMKTI |
| | | TNIYILNLAIADELFMLGLPFLAMQVALVHWPFGKAIC |
| | | RVVMTVDGINQFTSIFCLTVMSIDRYLAVVHPIKSAK |
| | | WRRPRTAKMITMAVWGVSLLVILPIMIYAGLRSNQW |
| | | GRSSCTINWPGESGAWYTGFIIYTFILGFLVPLTIICLCY |
| | | LFIIIKVKSSGIRVGSSKRKKSEKKVTRMVSIVVAVFIF |
| | | CWLPFYIFNVSSVSMAISPTPALKGMFDFVVVLTYAN |
| | | SCANPILYAFLSDNFKKSFQNVLCLVKVSGTDDGERS |
| | | DSKQDKSRLNETTETQRTLLNGDLQTSI |

*Mutated amino acids in I domain region are underlined and bolded.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, e.g., elements that are conjunctively present in some cases and disjunctively present in other cases. Multiple elements listed with “and/or” should be construed in the same fashion, e.g., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, e.g., the inclusion of at least one, but also including more than one of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (e.g., “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A method of cell therapy for treating cancer, comprising the steps of:
administering to a subject with carcinoma a population of CAR-T cells expressing a chimeric antigen receptor (CAR) comprising from N-terminus to C-terminus:
(i) an I domain of the α_{L} subunit of human lymphocyte function-associated antigen-1,
(ii) a transmembrane domain,
(iii) one or more co-stimulatory domains, and
(iv) an activation domain,
wherein the I domain comprises amino acids 128-311 of SEQ ID NO: 1, with one or more mutations selected from the group consisting of F292A, F292S, L289G, or F265S,
wherein the cancer is a carcinoma is selected from the group consisting of non-small cell lung carcinoma, small cell lung carcinoma, squamous cell lung carcinoma, large cell lung carcinoma, cervical carcinoma, hepatocellular carcinoma, renal carcinoma, bladder carcinoma, head and neck carcinoma, an adenocarcinoma thereof, and a squamous cell carcinoma thereof, and
wherein the carcinoma overexpresses ICAM-1, and the CAR T cells bind to and kill carcinoma cells overexpressing ICAM-1.

2. The method of claim 1, wherein the one or more mutations comprise;
(a) F292A;
(b) F265S and F292G; or
(c) F265S, F292G, and G311C.

3-8. (canceled)

9. The method of claim 1, wherein the transmembrane domain is positioned between the I domain and the one or more co-stimulatory domains, and wherein the transmembrane domain is from a cell surface receptor selected from the group consisting of an alpha, beta or zeta chain of a T cell receptor, CD8 alpha, CD28, ICOS, GITR, CD3 epsilon, CD45, CD4, CD5, CD8, CD9, CD16, CD22, CD33, CD37, CD64, CD80, CD86, CD134, CD137, CD154, CD271, TNFRSF19, Killer Cell Immunoglobulin-Like Receptor (KIR), and a combination thereof; optionally wherein the transmembrane domain comprises an amino acid sequence set forth in any one SEQ ID NOs: 4-7.

10. (canceled)

11. The method of claim 1, wherein the one or more co-stimulatory domains are from 4-1BB (CD 137), CD28, OX40, ICOS, GITR, CD27, CD30, CD40, DAP 10, DAP12, BAFFR, HVEM, ICAM-1, lymphocyte function-associated antigen-1 (LFA-1), CD2, CD5, CD7, CD287, LIGHT, NKG2C, NKG2D, SLAMF7, NKp80, NKp30, NKp44, NKp46, CD 160, B7-H3, or a ligand that specifically binds with CD83 or a combination thereof; optionally wherein the costimulatory domain comprises an amino acid sequence set forth in any one of SEQ ID NOs: 8-10.

12-14. (canceled)

15. The method of claim 1, wherein the activation domain is from CD3 zeta, common FcR gamma (FCER1G), Fc gamma RIIa, FcR beta, CD3 gamma, CD3 delta, CD3epsilon, CD5, CD22, CD79a, CD79b, CD278 (ICOS), FcεRI, or CD66d; optionally wherein the activation domain comprises the amino acid sequence of SEQ ID NO: 11.

16. (canceled)

17. The method of claim 1, wherein the CAR further comprises a hinge domain between the I domain and the transmembrane domain, and wherein the hinge domain is from CD8 alpha, CD28, or IgG4; optionally wherein the hinge domain comprises an amino sequence set forth in any one of SEQ ID NOs: 12-14.

18. (canceled)

19. The method of claim 1, wherein the CAR-T cells further express an epitope tag, optionally wherein the epitope tag is a c-myc tag.

20. The method of claim 1, wherein the CAR-T cells further express or are co-administered with one or more additional polypeptides; optionally wherein the one or more additional polypeptides comprise human somatostatin receptor 2 (SSTR2).

21. (canceled)

22. The method of claim 20, wherein the one or more additional polypeptides comprise: (i) an immune checkpoint inhibitor selected from the group consisting of pembrolizumab, ipilimumab, nivolumab, atezolizumab, and combinations thereof, or (ii) an anti-inflammatory cytokine selected from the group consisting of IL-4, IL-10, IL-11, IL-13, and combinations thereof, or (iii) a proinflammatory cytokine antagonist which targets IFNγ, IL-1, IL-6, IL-12, GM-CSF, and combinations thereof.

23-28. (canceled)

29. The method of claim 20, wherein the one or more additional polypeptides comprise a therapeutic antibody selected from the group consisting of abagovomab, adecatumumab, afutuzumab, alemtuzumab, altumomab, amatuximab, anatumomab, arcitumomab, bavituximab, bectumomab, bevacizumab, bivatuzumab, blinatumomab, brentuximab, cantuzumab, catumaxomab, cetuximab, citatuzumab, cixutumumab, clivatuzumab, conatumumab, daratumumab, drozitumab, duligotumab, dusigitumab, detumomab, dacetuzumab, dalotuzumab, ecromeximab, elotuzumab, ensituximab, ertumaxomab, etaracizumab, farietuzumab, ficlatuzumab, figitumumab, flanvotumab, futuximab, ganitumab, gemtuzumab, girentuximab, glembatumumab, ibritumomab, igovomab, imgatuzumab, indatuximab, inotuzumab, intetumumab, ipilimumab, iratumumab, labetuzumab, lexatumumab, lintuzumab, lorvotuzumab, lucatumumab, mapatumumab, matuzumab, milatuzumab, minretumomab, mitumomab, moxetumomab, namatumab, naptumomab, necitumumab, nimotuzumab, nofetumomab, ocaratuzumab, ofatumumab, obinutuzumab, olaratumab, onartuzumab, oportuzumab, oregovomab, panitumumab, parsatuzumab, patritumab, pemtumomab, pertuzumab, pintumomab, pritumumab, racotumomab, radretumab, rilotumumab, rituximab, robatumumab, satumomab, sibrotuzumab, siltuximab, simtuzumab, solitomab, tacatuzumab, taplitumomab, tenatumomab, teprotumumab, tigatuzumab, tositumomab, trastuzumab, tucotuzumab, ublituximab, veltuzumab, vorsetuzumab, votumumab, zalutumumab, CC49 and 3F8.

30. The method of claim 1, wherein the subject is additionally administered or infused with a second population of CAR-T cells expressing a second CAR, which comprises a c-Met binding domain, a CEACAM-5 binding domain, or an EpCAM binding domain.

31. (canceled)

32. The method of claim 1, wherein the CAR-T cells express SSTR2, and the method comprises the steps of:
(i) incubating the CAR-T cells with a radioactive label that binds to SSTR2 before administration into a patient with the carcinoma, wherein the radioactive label is ⁶⁸Ga-DOTATOC or ⁶⁸Ga-DOTATATE;
(ii) intravenously infusing the CAR-T cells labeled in step (i) into the patient; and
(iii) detecting the labeled CAR-T cell distribution in vivo by PET/CT imaging.

33-34. (canceled)

35. The method of any one of claim 1, further comprising the step of expanding the CAR-T cells in the presence of a tyrosine kinase inhibitor selected from the group consisting of dasatinib, ponatinib, saracatinib, bosutinib, nilotinib, and combinations thereof.

36-37. (canceled)

38. The method of claim 1, further comprising the step of transfecting an expression vector encoding the CAR to produce the CAR-T cells prior to the administration step, wherein the expression vector is a lentivirus comprising, from 5′ and 3, coding sequences encoding:
(i) the I domain;
(ii) a CD8 hinge region;
(iii) a CD28 transmembrane domain;
(iv) a CD28 costimulatory domain;
(v) a 4-1BB costimulatory domain; and
(vi) a CD3ζ activation domain.

39-41. (canceled)

42. The method of claim 38, wherein the expression vector further encodes an epitope tag, optionally wherein the epitope tag is upstream of the I domain and/or is a c-myc tag.

43. The method of claim 38, wherein the expression vector further comprises one or more self-cleaving 2A peptide coding sequences or IRES elements selected from the group consisting of porcine teschovirus-1 (P2A), equine rhinitis A virus (E2A), thosea asigna virus (T2A), or foot-and-mouth disease virus (F2A), and combinations thereof.

44-45. (canceled)

46. The method of claim 43 wherein the expression vector further encodes SSTR2.

47-48. (canceled)

49. The method of claim 38, wherein the expression vector comprises a nucleic acid comprising the nucleotide sequence set forth in SEQ ID NO: 25 or SEQ ID NO: 26.

50. (canceled)

51. The method of claim 1, further comprising administering a chemotherapy, an immunotherapy, a radiotherapy, or a surgery to reduce tumor burden in the subject prior to the cell therapy against cancer.

52. (canceled)

53. The method of claim 1, wherein prior to administration of the CAR-T cells, the subject receives a lymphodepleting treatment to condition the subject for the administration step, wherein the lymphodepleting treatment comprises administering to the subject one or more of fludarabine, cyclophosphamide, and bendamustine.

54. (canceled)

55. The method of claim 1, further comprising administering to the subject a tyrosine kinase inhibitor selected from the group consisting of dasatinib, ponatinib, saracatinib, bosutinib, nilotinib, and combinations thereof.

56-62. (canceled)

63. The method of claim 1, wherein the CAR-T cells express SSTR2, and wherein the subject is administered a safety switch comprising an SSTR2-targeted cytotoxic conjugate if unacceptable toxicity from the CAR-T cells is identified, the conjugate comprising a cytotoxic agent coupled to an SSTR2 targeting moiety by a linker,
wherein the targeting moiety is somatostatin, a somatostatin analog, octreotide, lanreotide, lutathera (¹⁷⁷Lu-DOTATATE), ⁹⁰Y-DOTATOC, Tyr³-octreotate (TATE), vapreotide, cyclo(AA-Tyr-DTrp-Lys-Thr-Phe) where AA is α-N-Me lysine or N-Me glutamic acid, pasireotide, lanreotide, seglitide, or an anti-SSTR2 antibody,
wherein the cytotoxic agent is a maytansinoid, a camptothecin, a dolastatin, an aurastatin, a pyrrolobenzodiazepine (PBD), a calicheamicin, a duocarmycin, a tubulolysin, an analogue thereof, or a combination thereof; optionally wherein the cytotoxic agent is PEN-221, and
wherein the linker is either: (i) a cleavable linker selected from the group consisting of maleimidocaproyl-L-valine-L-citrulline-p-aminobenzoyl carbamate (mc-VC-PABC) and maleimido-dPEG8-L-valine-L-alaline-p-aminobenzoyl carbamate (M-dPEG8-VA-PABC), or (ii) a non-cleavable linker selected from the group consisting of maleimidocaproyl (MC), succinimidyl-4-(N-maleimidomethyl)cyclohexane-1-carboxylate (SMCC), and thioether-containing linker.

64-70. (canceled)


SEQUENCE LISTING (31 sequences)

SEQ ID NO: 1 - protein, 1145 aa, Homo sapiens
YNLDVRGARSFSPPRAGRHFGYRVLQVGNGVIVGAPGEGNSTGSLYQCQSGTGHCLPVTLRGSNYTSKYLGMTLATDPTDGSILACDPGLSRTCDQNTYLSGLCYLFRQNLQGPMLQGRPGFQECIKGNVDLVFLFDGSMSLQPDEFQKILDFMKDVMKKLSNTSYQFAAVQFSTSYKTEFDFSDYVKWKDPDALLKHVKHMLLLTNTFGAINYVATEVFREELGARPDATKVLIIITDGEATDSGNIDAAKDIIRYIIGIGKHFQTKESQETLHKFASKPASEFVKILDTFEKLKDLFTELQKKIYVIEGTSKQDLTSFNMELSSSGISADLSRGHAVVGAVGAKDWAGGFLDLKADLQDDTFIGNEPLTPEVRAGYLGYTVTWLPSRQKTSLLASGAPRYQHMGRVLLFQEPQGGGHWSQVQTIHGTQIGSYFGGELCGVDVDQDGETELLLIGAPLFYGEQRGGRVFIYQRRQLGFEEVSELQGDPGYPLGRFGEAITALTDINGDGLVDVAVGAPLEEQGAVYIFNGRHGGLSPQPSQRIEGTQVLSGIQWFGRSIHGVKDLEGDGLADVAVGAESQMIVLSSRPVVDMVTLMSFSPAEIPVHEVECSYSTSNKMKEGVNITICFQIKSLYPQFQGRLVANLTYTLQLDGHRTRRRGLFPGGRHELRRNIAVTTSMSCTDFSFHFPVCVQDLISPINVSLNFSLWEEEGTPRDQRAQGKDIPPILRPSLHSETWEIPFEKNCGEDKKCEANLRVSFSPARSRALRLTAFASLSVELSLSNLEEDAYWVQLDLHFPPGLSFRKVEMLKPHSQIPVSCEELPEESRLLSRALSCNVSSPIFKAGHSVALQMMFNTLVNSSWGDSVELHANVTCNNEDSDLLEDNSATTIIPILYPINILIQDQEDSTLYVSFTPKGPKIHQVKHMYQVRIQPSIHDHNIPTLEAVVGVPQPPSEGPITHQWSVQMEPPVPCHYEDLERLPDAAEPCLPGALFRCPVVFRQEILVQVIGTLELVGEIEASSMFSLCSSLSISFNSSKHFHLYGSNASLAQVVMKVDVVYEKQMLYLYVLSGIGGLLLLLLIFIVLYKVGFFKRNLKEKMEAGRGVPNGIPAEDSEQLASGQEAGDPGCLKPLHEKDSESGGGKD

SEQ ID NO: 2 - protein, 184 aa, Homo sapiens
GNVDLVFLFDGSMSLQPDEFQKILDFMKDVMKKLSNTSYQFAAVQFSTSYKTEFDFSDYVKWKDPDALLKHVKHMLLLTNTFGAINYVATEVFREELGARPDATKVLIIITDGEATDSGNIDAAKDIIRYIIGIGKHFQTKESQETLHKFASKPASEFVKILDTFEKLKDLFTELQKKIYVIEG

SEQ ID NO: 3 - protein, 184 aa, Homo sapiens
GNVDLVFLFDGSMSLQPDEFQKILDFMKDVMKKLSNTSYQFAAVQFSTSYKTEFDFSDYVKWKDPDALLKHVKHMLLLTNTFGAINYVATEVFREELGARPDATKVLIIITDGEATDSGNIDAAKDIIRYIIGIGKHFQTKESQETLHKFASKPASEFVKILDTAEKLKDLFTELQKKIYVIEG

SEQ ID NO: 4 - protein, 24 aa, Homo sapiens
IYIWAPLAGTCGVLLLSLVITLYC

SEQ ID NO: 5 - protein, 24 aa, Homo sapiens
FWVLVVVGGVLACYSLLVTVAFII

SEQ ID NO: 6 - protein, 24 aa, Homo sapiens
FWLPIGCAAFVVVCILGCILICWL

SEQ ID NO: 7 - protein, 25 aa, Homo sapiens
LGWLTVVLLAVAACVLLLTSAQLGL

SEQ ID NO: 8 - protein, 42 aa, Homo sapiens
KRGRKKLLYIFKQPFMRPVQTTQEEDGCSCRFPEEEEGGCEL

SEQ ID NO: 9 - protein, 41 aa, Homo sapiens
RSKRSRLLHSDYMNMTPRRPGPTRKHYQPYAPPRDFAAYRS

SEQ ID NO: 10 - protein, 42 aa, Homo sapiens
ALYLLRRDQRLPPDAHKPPGGGSFRTPIQEEQADAHSTLAKI

SEQ ID NO: 11 - protein, 112 aa, Homo sapiens
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 12 - protein, 45 aa, Homo sapiens
TTTPAPRPPTPAPTIASQPLSLRPEACRPAAGGAVHTRGLDFACD

SEQ ID NO: 13 - protein, 47 aa, Homo sapiens
RAAAIEVMYPPPYLDNEKSNGTIIHVKGKHLCPSPLFPGPSKPKDPK

SEQ ID NO: 14 - protein, 11 aa, Homo sapiens
SKYGPPCPSCP

SEQ ID NO: 15 - protein, 12 aa, Homo sapiens
GSEQKLISEEDL

SEQ ID NO: 16 - protein, 369 aa, Homo sapiens
MDMADEPLNGSHTWLSIPFDLNGSVVSTNTSNQTEPYYDLTSNAVLTFIYFVVCIIGLCGNTLVIYVILRYAKMKTITNIYILNLAIADELFMLGLPFLAMQVALVHWPFGKAICRVVMTVDGINQFTSIFCLTVMSIDRYLAVVHPIKSAKWRRPRTAKMITMAVWGVSLLVILPIMIYAGLRSNQWGRSSCTINWPGESGAWYTGFIIYTFILGFLVPLTIICLCYLFIIIKVKSSGIRVGSSKRKKSEKKVTRMVSIVVAVFIFCWLPFYIFNVSSVSMAISPTPALKGMFDFVVVLTYANSCANPILYAFLSDNFKKSFQNVLCLVKVSGTDDGERSDSKQDKSRLNETTETQRTLLNGDLQTSI

SEQ ID NO: 17 - protein, 314 aa, Homo sapiens
MDMADEPLNGSHTWLSIPFDLNGSVVSTNTSNQTEPYYDLTSNAVLTFIYFVVCIIGLCGNTLVIYVILRYAKMKTITNIYILNLAIADELFMLGLPFLAMQVALVHWPFGKAICRVVMTVDGINQFTSIFCLTVMSIDRYLAVVHPIKSAKWRRPRTAKMITMAVWGVSLLVILPIMIYAGLRSNQWGRSSCTINWPGESGAWYTGFIIYTFILGFLVPLTIICLCYLFIIIKVKSSGIRVGSSKRKKSEKKVTRMVSIVVAVFIFCWLPFYIFNVSSVSMAISPTPALKGMFDFVVVLTYANSCANPILYAF

SEQ ID NO: 18 - protein, 21 aa, Homo sapiens
MALPVTALLLPLALLLHAARP

SEQ ID NO: 19 - protein, 4 aa, synthetic construct
GGGS

SEQ ID NO: 20 - protein, 15 aa, synthetic construct
GGGGSGGGGSGGGGS

SEQ ID NO: 21 - protein, 22 aa, Teschovirus A
GSGATNFSLLKQAGDVEENPGP

SEQ ID NO: 22 - protein, 23 aa, Equine rhinitis A virus
GSGQCTNYALLKLAGDVESNPGP

SEQ ID NO: 23 - protein, 21 aa, Thosea asigna virus
GSGEGRGSLLTCGDVEENPGP

SEQ ID NO: 24 - protein, 25 aa, Foot-and-mouth disease virus
GSGVKQTLNFDLLKLAGDVESNPGP

SEQ ID NO: 25 - DNA, 1359 bp, synthetic construct
  source 1..1359: mol_type = other DNA
ggcaacgtagacctggtatttctgtttgatggttcgatgagcttgcagccagatgaatttcagaaaattctggacttcatgaaggatgtgatgaagaaactcagcaacacttcgtaccagtttgctgctgttcagttttccacaagctacaaaacagaatttgatttctcagattatgttaaatggaaggaccctgatgctctgctgaagcatgtaaagcacatgttgctgttgaccaatacctttggtgccatcaattatgtcgcgacagaggtgttccgggaggagctgggggcccggccagatgccaccaaagtgcttatcatcatcacggatggggaggccactgacagtggcaacatcgatgcggccaaagacatcatccgctacatcatcgggattggaaagcattttcagaccaaggagagtcaggagaccctccacaaatttgcatcaaaacccgcgagcgagtttgtgaaaattctggacacagctgagaagctgaaagatctattcactgagctgcagaagaagatctatgtcattgagggcgctagcacgaccactccggcgccgcgcccaccgactccggccccaactatcgcgagccagcccctgtcgctgaggccggaagcatgccgccctgccgccggaggtgctgtgcatacccggggattggacttcgcatgcgacttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaagcggggtcggaaaaagcttctgtacattttcaagcagcccttcatgaggcccgtgcaaaccacccaggaggaggacggttgctcctgccggttccccgaagaggaagaaggaggttgcgagctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 26 - DNA, 2652 bp, synthetic construct
  source 1..2652: mol_type = other DNA
atggccttaccagtgaccgccttgctcctgccgctggccttgctgctccacgccgccaggccgggatccgaacaaaaactcatctcagaagaggatctgggatcgggcaacgtagacctggtatttctgtttgatggttcgatgagcttgcagccagatgaatttcagaaaattctggacttcatgaaggatgtgatgaagaaactcagcaacacttcgtaccagtttgctgctgttcagttttccacaagctacaaaacagaatttgatttctcagattatgttaaatggaaggaccctgatgctctgctgaagcatgtaaagcacatgttgctgttgaccaatacctttggtgccatcaattatgtcgcgacagaggtgttccgggaggagctgggggcccggccagatgccaccaaagtgcttatcatcatcacggatggggaggccactgacagtggcaacatcgatgcggccaaagacatcatccgctacatcatcgggattggaaagcattttcagaccaaggagagtcaggagaccctccacaaatttgcatcaaaacccgcgagcgagtttgtgaaaattctggacacagctgagaagctgaaagatctattcactgagctgcagaagaagatctatgtcattgagggcgctagcacgaccactccggcgccgcgcccaccgactccggccccaactatcgcgagccagcccctgtcgctgaggccggaagcatgccgccctgccgccggaggtgctgtgcatacccggggattggacttcgcatgcgacttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaagcggggtcggaaaaagcttctgtacattttcaagcagcccttcatgaggcccgtgcaaaccacccaggaggaggacggttgctcctgccggttccccgaagaggaagaaggaggttgcgagctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcactagtggaagcggagctactaacttcagcctgctgaagcaggctggagacgtggaggagaaccctggaccttctagaatggacatggcggatgagccactcaatggaagccacacatggctatccattccatttgacctcaatggctctgtggtgtcaaccaacacctcaaaccagacagagccgtactatgacctgacaagcaatgcagtcctcacattcatctattttgtggtctgcatcattgggttgtgtggcaacacacttgtcatttatgtcatcctccgctatgccaagatgaagaccatcaccaacatttacatcctcaacctggccatcgcagatgagctcttcatgctgggtctgcctttcttggctatgcaggtggctctggtccactggccctttggcaaggccatttgccgggtggtcatgactgtggatggcatcaatcagttcaccagcatcttctgcctgacagtcatgagcatcgaccgatacctggctgtggtccaccccatcaagtcggccaagtggaggagaccccggacggccaagatgatcaccatggctgtgtggggagtctctctgctggtcatcttgcccatcatgatatatgctgggctccggagcaaccagtgggggagaagcagctgcaccatcaactggccaggtgaatctggggcttggtacacagggttcatcatctacactttcattctggggttcctggtacccctcaccatcatctgtctttgctacctgttcattatcatcaaggtgaagtcctctggaatccgagtgggctcctctaagaggaagaagtctgagaagaaggtcacccgaatggtgtccatcgtggtggctgtcttcatcttctgctggcttcccttctacatattcaacgtttcttccgtctccatggccatcagccccaccccagcccttaaaggcatgtttgactttgtggtggtcctcacctatgctaacagctgtgccaaccctatcctatatgccttcttgtctgacaacttcaagaagagcttccagaatgtcctctgcttggtcaaggtgagcggcacagatgatggggagcggagtgacagtaagcaggacaaatcccggctgaatgagaccacggagacccagaggaccctcctcaatggagacctccaaaccagtatctaa

SEQ ID NO: 27 - protein, 453 aa, synthetic construct
GNVDLVFLFDGSMSLQPDEFQKILDFMKDVMKKLSNTSYQFAAVQFSTSYKTEFDFSDYVKWKDPDALLKHVKHMLLLTNTFGAINYVATEVFREELGARPDATKVLIIITDGEATDSGNIDAAKDIIRYIIGIGKHFQTKESQETLHKFASKPASEFVKILDTAEKLKDLFTELQKKIYVIEGASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 28 - protein, 848 aa, synthetic construct
GNVDLVFLFDGSMSLQPDEFQKILDFMKDVMKKLSNTSYQFAAVQFSTSYKTEFDFSDYVKWKDPDALLKHVKHMLLLTNTFGAINYVATEVFREELGARPDATKVLIIITDGEATDSGNIDAAKDIIRYIIGIGKHFQTKESQETLHKFASKPASEFVKILDTAEKLKDLFTELQKKIYVIEGASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRTSGSGATNFSLLKQAGDVEENPGPSRMDMADEPLNGSHTWLSIPFDLNGSVVSTNTSNQTEPYYDLTSNAVLTFIYFVVCIIGLCGNTLVIYVILRYAKMKTITNIYILNLAIADELFMLGLPFLAMQVALVHWPFGKAICRVVMTVDGINQFTSIFCLTVMSIDRYLAVVHPIKSAKWRRPRTAKMITMAVWGVSLLVILPIMIYAGLRSNQWGRSSCTINWPGESGAWYTGFIIYTFILGFLVPLTIICLCYLFIIIKVKSSGIRVGSSKRKKSEKKVTRMVSIVVAVFIFCWLPFYIFNVSSVSMAISPTPALKGMFDFVVVLTYANSCANPILYAFLSDNFKKSFQNVLCLVKVSGTDDGERSDSKQDKSRLNETTETQRTLLNGDLQTSI

SEQ ID NO: 29 - protein, 883 aa, synthetic construct
MALPVTALLLPLALLLHAARPGSEQKLISEEDLGSGNVDLVFLFDGSMSLQPDEFQKILDFMKDVMKKLSNTSYQFAAVQFSTSYKTEFDFSDYVKWKDPDALLKHVKHMLLLTNTFGAINYVATEVFREELGARPDATKVLIIITDGEATDSGNIDAAKDIIRYIIGIGKHFQTKESQETLHKFASKPASEFVKILDTAEKLKDLFTELQKKIYVIEGASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRTSGSGATNFSLLKQAGDVEENPGPSRMDMADEPLNGSHTWLSIPFDLNGSVVSTNTSNQTEPYYDLTSNAVLTFIYFVVCIIGLCGNTLVIYVILRYAKMKTITNIYILNLAIADELFMLGLPFLAMQVALVHWPFGKAICRVVMTVDGINQFTSIFCLTVMSIDRYLAVVHPIKSAKWRRPRTAKMITMAVWGVSLLVILPIMIYAGLRSNQWGRSSCTINWPGESGAWYTGFIIYTFILGFLVPLTIICLCYLFIIIKVKSSGIRVGSSKRKKSEKKVTRMVSIVVAVFIFCWLPFYIFNVSSVSMAISPTPALKGMFDFVVVLTYANSCANPILYAFLSDNFKKSFQNVLCLVKVSGTDDGERSDSKQDKSRLNETTETQRTLLNGDLQTSI

SEQ ID NO: 30 - protein, 862 aa, synthetic construct
GSEQKLISEEDLGSGNVDLVFLFDGSMSLQPDEFQKILDFMKDVMKKLSNTSYQFAAVQFSTSYKTEFDFSDYVKWKDPDALLKHVKHMLLLTNTFGAINYVATEVFREELGARPDATKVLIIITDGEATDSGNIDAAKDIIRYIIGIGKHFQTKESQETLHKFASKPASEFVKILDTAEKLKDLFTELQKKIYVIEGASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRTSGSGATNFSLLKQAGDVEENPGPSRMDMADEPLNGSHTWLSIPFDLNGSVVSTNTSNQTEPYYDLTSNAVLTFIYFVVCIIGLCGNTLVIYVILRYAKMKTITNIYILNLAIADELFMLGLPFLAMQVALVHWPFGKAICRVVMTVDGINQFTSIFCLTVMSIDRYLAVVHPIKSAKWRRPRTAKMITMAVWGVSLLVILPIMIYAGLRSNQWGRSSCTINWPGESGAWYTGFIIYTFILGFLVPLTIICLCYLFIIIKVKSSGIRVGSSKRKKSEKKVTRMVSIVVAVFIFCWLPFYIFNVSSVSMAISPTPALKGMFDFVVVLTYANSCANPILYAFLSDNFKKSFQNVLCLVKVSGTDDGERSDSKQDKSRLNETTETQRTLLNGDLQTSI

SEQ ID NO: 31 - protein, 848 aa, synthetic construct; identical to SEQ ID NO: 28